(19) United States
(12) Patent Application Publication
(10) Pub. No.: US 2021/0164971 A1
(43) Pub. Date: Jun. 3, 2021

(54) DROPLET ARRAYS FOR DETECTION AND QUANTIFICATION OF ANALYTES

(71) Applicants: The Brigham and Women's Hospital, Inc., Boston, MA (US); President and Fellows of Harvard College, Cambridge, MA (US)

(72) Inventors: David R. Walt, Boston, MA (US); Limor Cohen, Boston, MA (US); David A. Weitz, Bolton, MA (US); Nai Wen Cui, Cambridge, MA (US); Yamei Cai, Cambridge, MA (US)

(21) Appl. No.: 17/267,827

(22) PCT Filed: Aug. 15, 2019
(86) PCT No.: PCT/US2019/046665

§ 371 Date: Feb. 11, 2021

Related U.S. Application Data
(60) Provisional application No. 62/765,074, filed on Aug. 17, 2018.

(51) Int. Cl. G01N 33/543; G01N 33/58

(52) U.S. Cl. CPC G01N 33/54333; G01N 2333/55; G01N 2333/57; G01N 33/581

(57) ABSTRACT

The invention provides high-sensitivity methods for detection and quantification of target analytes in liquid samples (e.g., biological or environmental samples). The methods can be multiplexed to allow simultaneous detection and quantification of multiple target analytes that are contained in the same sample. The invention also provides related compositions and kits.

### RELATED APPLICATIONS

The instant application claims priority to U.S. Provisional Application No. 62/765,074, filed on Aug. 17, 2018, the entire contents of which are expressly incorporated herein by reference.

### STATEMENT AS TO FEDERALLY FUNDED RESEARCH

This invention was made with government support under grant numbers DMR1708729 and DMR1420570 awarded by the National Science Foundation (NSF). The government has certain rights in this invention.

### FIELD OF THE INVENTION

The invention relates to methods and compositions for detection and quantification of analytes.

### BACKGROUND OF THE INVENTION

Biomarker measurements in biological fluids are important for disease detection, monitoring, and treatment. Biomarkers (e.g., nucleic acids and proteins) are typically elevated in the affected organ but become diluted and decrease in concentration once they enter the bloodstream or other biological fluids (e.g., saliva and cerebrospinal fluid). The ability to detect low levels of biomarkers is expected to lead to early detection of disease and increased survival rates. Sensitive nucleic acid detection is achieved by using the polymerase chain reaction (PCR) and related technologies, which can amplify a single molecule. Such amplification approaches do not currently exist for proteins, and protein detection methods can suffer from a lack of analytical sensitivity.

A gold-standard tool for detecting and quantifying proteins in biological fluids is the enzyme linked immunosorbent assay (ELISA). In a standard sandwich ELISA, a capture antibody specific to a target protein is adsorbed onto the surface of a microtiter plate. The biological sample is then incubated with the capture antibody, and the target protein binds to the capture antibody. A detection antibody that is conjugated to biotin is then added. The detection antibody recognizes an epitope of the target protein distinct from the epitope recognized by the capture antibody. An enzyme that can bind to the biotinylated detection antibody via biotin-streptavidin interaction is added followed by a fluorogenic substrate. The enzyme turns over the substrate molecules to produce a fluorescent product. The fluorescence intensity is correlated to the concentration of the target protein. The traditional ELISA suffers from lack of analytical sensitivity. A standard reaction volume of an ELISA is 50 μL to 100 μL. The fluorescent product diffuses into a large volume and therefore an enzyme must turn over millions of substrate molecules to generate a detectable signal above the background. This results in low analytical sensitivity and inability to measure many potentially important proteins in biological samples.

Thus, there remains a need in the art for high-sensitivity methods for detection and quantification of target analytes.

### SUMMARY OF THE INVENTION

The invention provides improved methods and compositions for detection and quantification of analytes. By capturing and detecting target analytes in single droplets with high efficiency and minimal sample loss, the methods of the current invention improve the sensitivity of detection by at least one order of magnitude, as compared to prior single molecule detection methods. The methods are particularly suitable for detection and quantification of target analytes with ultra-low concentrations, e.g., molecules in biological samples.

Without being limited by theory, it is believed that the current invention increases the sensitivity of detection and quantification of target analytes by interrogating a large ensemble of droplets all at once, and interrogating a large percentage of droplets such as at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 100% of the droplets.

In one aspect, the invention provides a method of detecting a target analyte in a sample that includes the steps of: (a) contacting a sample containing or suspected of containing a target analyte with a plurality of capture probes, the capture probes being linked to one or more capture ligands that specifically bind to the target analyte, and incubating to allow binding of the capture ligands to the target analytes; (b) contacting the product of (a) with a plurality of detection probes that specifically bind to the target analyte, and incubating to allow binding of the detection probes to the target analyte, the detection probes each being linked to a detectable moiety; (c) producing a plurality of droplets from the product of (b); and (d) detecting the detectable moieties present in the plurality of droplets, thereby detecting the target analyte in the sample. Steps (a) and (b) can be performed sequentially or simultaneously. In some embodiments, steps (a) and (b) are performed sequentially. In other embodiments, steps (a) and (b) are performed simultaneously.

In another aspect, the invention provides a method of detecting a target analyte in a sample that includes the steps of: (a) contacting a sample containing or suspected of containing a target analyte with: (i) a plurality of detection probes that specifically bind to the target analyte, and (ii) a plurality of capture probes, the capture probes being linked to one or more immobilized target analytes, wherein the detection probes competitively bind to the target analytes contained in the sample and to the immobilized target analytes; (b) incubating the product of step (a) to allow binding of the detection probes to the target analytes contained in the sample or to the immobilized target analytes; (c) labeling the detection probes that are bound to the immobilized target analytes linked to the capture probes of step (b) with detectable moieties; (d) producing a plurality of droplets of the product of step (c); and (e) detecting the detectable moieties in the droplets, thereby detecting the target analyte in the sample. Steps (a), (b), and/or (c) can be performed sequentially or simultaneously. In some embodiments, steps (a), (b), and/or (c) are performed sequentially. In other embodiments, steps (a), (b), and/or (c) are performed simultaneously.

In some embodiments of any of the methods of the invention, all or substantially all of the droplets contain zero or one capture probes. In other embodiments, all or substantially all of the droplets contain more than one capture probe.

In some embodiments of any of the methods of the invention, the capture probes are linked to about 1 to about 10¹² capture ligands, e.g., about 1, about 10¹, about 10³, about 10⁴, about 10⁵, about 10⁶, about 10⁷, about 10⁸, about 10⁹, about 10¹⁰, about 10¹¹, or about 10¹².

In some embodiments of any of the methods of the invention, the concentration of the target analyte in the sample ranges from about 0 aM to about 1 mM, e.g., about 1 aM to about 1 mM.

In some embodiments of any of the methods of the invention, the droplets have a volume of about 0.01 pL to about 10 nL.

In some embodiments of any of the methods of the invention, producing a plurality of droplets is performed using a microfluidic device. In some embodiments, the microfluidic device is a flow-focusing device.

In some embodiments of any of the methods of the invention, the capture ligand and/or the detection probe is an antibody, an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a polypeptide, a nucleic acid, a molecularly-imprinted polymer, a receptor, or a small molecule. In some embodiments, the antibody is a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb).

In some embodiments of any of the methods of the invention, the capture probes are selected from the group consisting of beads, nanotubes, or polymers. In some embodiments, the beads are paramagnetic beads, silica beads, or hydrogel beads. In some embodiments, the beads have a size of about 1 μm to about 50 μm.

In some embodiments of any of the methods of the invention, the sample is contacted with about 10,000 to about 100,000,000 capture probes, e.g., in step (a).

In some embodiments of any of the methods of the invention, the detectable moiety is or includes an enzymatic label, a fluorescent label, a radioactive label, or a metal label. In some embodiments, the detectable moiety is or includes an enzymatic label (e.g., beta-galactosidase, horseradish peroxidase, glucose oxidase, or alkaline phosphatase). In some embodiments, the plurality of droplets is formed with an enzyme substrate. In some embodiments, the enzyme substrate is pre-mixed with the capture probes in step (a). In some embodiments, the enzyme substrate is di-β-D-galatopyranoside (FDG).

In some embodiments of any of the methods of the invention, producing the droplets includes mixing, e.g., by chaotic advection. In some embodiments, the mixing by chaotic advection is performed using channels with turns in a microfluidic device.

In some embodiments of any of the methods of the invention, the droplets include a density gradient medium.

In some embodiments of any of the methods of the invention, the sample includes a biological sample (e.g., a body fluid) or an environmental sample. In some embodiments, the biological sample includes a body fluid, e.g., lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, or spinal fluid. In other embodiments, the biological sample is a breast tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate.

In some embodiments of any of the methods of the invention, the target analyte is a protein, a nucleic acid (e.g., a modified nucleic acid or an miRNA), a polysaccharide, a lipid, an extracellular vesicle, a glycan, a toxin, a cell, a fatty acid, a therapeutic agent, a pathogen, an organism, a virus, or a small molecule.

In some embodiments of any of the methods of the invention, the detection includes single-molecule detection of the detectable moieties.

In some embodiments of any of the methods of the invention, the method further includes detecting an additional target analyte (e.g., a protein, a nucleic acid, a polysaccharide, a lipid, a cell, a fatty acid, a therapeutic agent, an organism, a virus, or a small molecule) in the sample.

In another aspect, the invention provides a method of detecting a first target analyte and a second target analyte in a sample that includes the steps of: (a) contacting a sample containing or suspected of containing a first target analyte and a second target analyte with: (i) a plurality of first capture probes, the first capture probes being linked to one or more first capture ligands that specifically bind to the first target analyte; and (ii) a plurality of second capture probes, the second capture probes being linked to one or more second capture ligands that specifically bind to the second target analyte, and incubating to allow binding of the first and second capture ligands to the first and second target analytes, respectively; (b) contacting the product of (a) with: (i) a plurality of first detection probes that specifically bind to the first target analyte, and (ii) a plurality of second detection probes that specifically bind to the second target analyte, and incubating to allow binding of the first and second detection probes to the first and second target analytes, respectively, wherein the first and second detection probes are each labelled with a detectable moiety; (c) producing a plurality of droplets from the product of (b); and (d) detecting the detectable moieties present in the plurality of droplets, thereby detecting the first target analyte and the second target analyte in the sample. In some embodiments, the first capture probe and the second capture probe are detectably and distinguishably labeled, and step (d) includes detecting the capture probes and the detectable moieties present in the plurality of droplets. In some embodiments, the first capture probe is labelled with a first dye, and the second capture probe is labelled with a second dye. In some embodiments, the first detection probe is labelled with a first detectable moiety, and the second detection probe is labelled with a second detectable moiety, and the first detectable moiety and the second detectable moiety are distinguishable. In other embodiments, the first detection probe is labelled with a first detectable moiety, and the second detection probe is labelled with a second detectable moiety, and the first detectable moiety and the second detectable moiety are not distinguishable.

In some embodiments of any of the methods of the invention, all or substantially all of the droplets contain zero or one target analyte molecule.

In some embodiments of any of the methods of the invention, the detectable moieties are detected in at least 30% of the droplets. In some embodiments of any of the methods of the invention, the detectable moieties are detected in at least 40% of the droplets. In some embodiments of any of the methods of the invention, the detectable moieties are detected in at least 50% of the droplets. In some embodiments of any of the methods of the invention, the detectable moieties are detected in at least 60% of the droplets. In some embodiments of any of the methods of the invention, the detectable moieties are detected in at least 70% of the droplets. In some embodiments of any of the methods of the invention, the detectable moieties are detected in at least 80% of the droplets.

Other features and advantages of the invention will be apparent from the following Detailed Description, the drawings, and the claims.

### BRIEF DESCRIPTION OF THE DRAWINGS

FIG. 1A is a schematic diagram depicting the formation of immunocomplexes of antibody-coated capture beads and target analytes. The antibody-coated capture beads are added in excess to a sample containing low concentrations of target analytes. The immunocomplexes are then incubated with a detection antibody (e.g., a biotinylated antibody) and an enzyme (e.g., a streptavidin-β-galactosidase), forming an enzyme-labeled immunocomplex.

FIG. 1B is a schematic diagram depicting the reconstitution of the immunocomplexes in an enzyme substrate.

FIG. 1C is a schematic diagram depicting that the immunocomplexes are loaded into pL droplets such that each droplet contains zero or one immunocomplex based on the Poisson distribution.

FIG. 1D is a schematic diagram showing that the droplets are loaded onto a chamber, foring droplet arrays.

FIG. 1E depicts images obtained in three channels that identify the droplets containing the target analytes, droplets containing the beads, and the droplets, from left to right, respectively.

FIG. 2A is a bar graph showing average number of enzymes per bead (AEB) at various concentrations of target analyte using different numbers of beads. For a given concentration of target analyte, as the number of beads increases, the AEB decreases.

FIG. 2B is a bar graph showing the digital measurement of number of positive events at various concentrations of target analytes when different percentages of beads are analyzed. As the percentage of beads analyzed increases, the number of positive events increases.

FIG. 2C depicts the theoretical limits of detection (LODs) at different binding affinities (KDs).

FIG. 3 is a micrograph of a flow-focusing device for droplet generation. The inlet channel for beads is labelled A, the inlet channel for the enzyme substrate is labelled B, and the inlet channel for oil is labelled C.

FIG. 4 is a series of images showing a droplet microfluidic device setup.

FIG. 5 is a micrograph of a droplet array capable of housing one million droplets.

FIG. 6A is a series of micrographs showing a white light image (left panel) and a fluorescent image (right panel) of a droplet array.

FIG. 6B is a series of micrographs showing a zoomed-in view of FIG. 6A. The top panel is a white light image. Beads inside droplets are clearly visible. The bottom panel is a fluorescent image showing the florescent signal in bead-containing droplets.

FIG. 7A depicts a white light image of beads, FIG. 7B depicts a fluorescence image (DAPI) of the beads, and FIG. 8C depicts a fluorescence image (CY® 7) of the beads.

FIG. 8A depicts channels to promote mixing of droplet contents. FIG. 8B depicts the signal of enzyme substrate (FDG). Single molecule detection of enzymes was achieved. FIG. 8C depicts the signal of dye-encoded beads (4′,6-diamidino-2-phenylindole (DAPI)).

FIGS. 9A-9C are a panel of micrographs showing a device configuration in which the beads were pre-mixed with the substrate and then loaded onto the device for encapsulation inside droplets. FIG. 9A shows an image of the device containing beads in substrate (labeled with an “a”) and oil (labeled with a “b”). FIG. 9B shows the signal of the enzyme substrate (RGP). Each droplet contains multiple enzymes per bead. FIG. 9C shows the signal of dye-encoded beads (DAPI).

FIG. 10A is a schematic illustration of the design of a device with two inlets (left), one for the oil with surfactant (A) and one for the beads with substrate mixture (B). The outlet (C) is used to collect the formed droplets. The chamber for droplet arrays (right) contains an inlet (B) and an outlet (A).

FIG. 10B depicts the blocking posts that are used to prevent droplets from escaping. The distance between two posts is 7 μm. Spacing between two posts is 15 μm, and the post diameter is 60 μm. This allows the droplets to pass through.

FIG. 11 is a panel of microscopic images depicting the detection of beads and target analytes contained in single droplets, at different concentrations of target analytes.

FIG. 12A is a panel of histograms showing the signal intensity of each droplet that contains a bead at various concentrations of interferon γ (IFNγ).

FIG. 12B is a panel of histograms showing the signal intensity of each droplet that contains a bead at various concentrations of interleukin 2 (IL-2).

FIG. 13A depicts calibration curves for the droplet-based assay (Droplet Simoa) for IFNγ and IL-2.

FIG. 13B depicts calibration curves for the Simoa assay using the HD-1 analyzer (Simoa) for IFNγ and IL-2.

FIG. 13C (top panel) depicts signal over background for the calibration curves for IFNγ and IL-2 for both the Droplet Simoa and Simoa assays. Zoomed in view (bottom panel) depicts from 0.0001 fM to 1 fM for IFNγ and 0.001 fM to 1 fM for IL-2 for both the Droplet Simoa and Simoa assays (bottom). Error bars represent replicate measurements.

FIG. 14 depicts measurements of endogenous proteins in serum samples. IFNγ and IL-2 levels were measured in serum using both the droplet-based Simoa method described herein and the Simoa HD-1 Analyzer. Concentrations shown are measured values and are not corrected for the serum sample dilution factor.

### DETAILED DESCRIPTION OF THE INVENTION

The invention provides methods and compositions for detection or measuring the concentration of a target analyte. The invention is based, at least in part, on the discovery that droplet arrays can be used for ultra-sensitive detection of target analytes. The methods described herein increase the sensitivity of detection by at least one order of magnitude, as compared to prior single molecule methods. Furthermore, the methods can be multiplexed for simultaneous detection of multiple target analytes in a single sample.

To overcome the low analytical sensitivity of traditional ELISA approaches, we previously developed an ultra-sensitive protein detection method using Single Molecule Arrays (e.g., SIMOA™) (see, for example, U.S. Pat. No. 8,236,574). SIMOA™ is a bead-based sandwich immunoassay in which single protein molecules are labeled with an enzyme and isolated inside femtoliter-sized wells. Briefly, in a SIMOA™ immunoassay, antibody-coated capture beads are added in excess to a sample containing low concentrations of target analyte molecules. Poisson statistics dictate that either one or zero target protein molecules will bind to each bead. The beads are then incubated with a biotinylated detection antibody and streptavidin-B-galactosidase, forming an enzyme-labeled immunocomplex. The beads are then loaded onto an array of 50 fL sized wells in which each well can hold only one bead. A fluorogenic substrate is added and the wells are sealed with oil, producing a locally high concentration of fluorescent product, enabling single molecule detection by counting active wells. At high protein concentrations, fluorescence intensity of the array is used to determine target concentration, thereby extending the dynamic range of the assay. The signal output is measured using the standard unit of average enzymes per bead (AEB). SIMOA™ assays can also be multiplexed to measure multiple proteins simultaneously in a biological sample.

To enhance the sensitivity of single molecule array (e.g., SIMOA™) assays and to make them more amenable to miniaturization and field or point-of-care use, we developed a droplet microfluidic-based assay, in which single protein molecules are isolated inside droplets. In a typical single molecule array assay, at least 500,000 beads are incubated with the sample and then loaded onto an array of 216,000 wells by gravity. Due to inefficiencies in bead loading, less than 5% of the beads are analyzed. The advantages of a droplet microfluidic-based assay include the ability to reduce the total number of beads in the assay and increase the number of beads that are analyzed.

Additional advantages of the droplet microfluidic-based assay include low cost and amenability with portable devices and automated instrumentation. Encapsulation of beads in droplets is fast, allowing rapid turnaround time from sample to results. The design of the devices described herein is simple and therefore the fabrication process can be scaled up easily. The devices can be made of inexpensive materials such as PDMS, as exemplified in the Examples below, but can also be made of other inexpensive materials such as glass or polymers for large-scale commercial manufacturing. In addition, the reagents for droplet generation are low in cost and easily available. Finally, the devices can be reused several times.

The methods disclosed herein are also amenable to other single molecule studies that are not based on bead-based immunoassays. One example is detection of rare enzyme molecules in blood.

### I. Definitions

As used herein, the term “about” refers to a value that is within 10% above or below the value being described.

The term “droplet” refers to an isolated portion of a first fluid that is completely surrounded by a second fluid. The droplet may be spherical or substantially spherical, or may assume other shapes as well. In most, but not all embodiments, the droplet and the fluid containing the droplet are substantially immiscible. In some cases, however, the droplet and the fluid containing the droplet may be miscible. In some cases, a hydrophilic liquid may be suspended in a hydrophobic liquid, a hydrophobic liquid may be suspended in a hydrophilic liquid, a gas bubble may be suspended in a liquid, and the like. Examples of hydrophilic liquids include, e.g., water and other aqueous solutions comprising water, such as cell or biological media, salt solutions, and the like. Examples of hydrophobic liquids include, e.g., oils such as hydrocarbons, silicon oils, fluorocarbon oils, organic solvents, and the like. Those of ordinary skill in the art can select suitable substantially miscible or substantially immiscible fluids, using contact angle measurements or the like, to carry out the techniques of the invention.

The term “fluid” refers to a liquid or a gas. A fluid cannot maintain a defined shape and will flow to fill the container in which it is placed. The fluid may have any suitable viscosity that permits flow.

The term “microfluidic,” as used herein, refers to a device, apparatus, or system including at least one fluid channel having a cross-sectional dimension of less than 1 mm, and a ratio of length to largest cross-sectional dimension of at least about 3:1. A “microfluidic channel,” as used herein, is a channel meeting these criteria. The “cross-sectional dimension” of the channel is measured perpendicular to the direction of fluid flow. In some embodiments, the fluid channels may be formed in part by a single component (e.g., an etched substrate or molded unit). Of course, larger channels, tubes, chambers, reservoirs, and the like can be used to store fluids in bulk and to deliver fluids to components of the devices used herein. In one set of embodiments, the maximum cross-sectional dimension of the channel(s) are less than 1 mm, less than 500 microns, less than 200 microns, less than 100 microns, less than 50 microns, or less than 25 microns. The dimensions of the channel may be chosen such that fluid is able to freely flow through the channel. The dimensions of the channel may also be chosen, for example, to allow a certain volumetric or linear flowrate of fluid in the channel. The number of channels and the shape of the channels can be varied by any method known to those of ordinary skill in the art. In some cases, more than one channel may be used. For example, two or more channels (e.g., two, three, four, five, six, seven, eight, nine, ten, or more) may be used, where they are positioned inside each other, positioned adjacent to each other, positioned to intersect with each other, and the like.

A “channel,” as used herein, means a feature on or in a device, apparatus, or system that at least partially directs the flow of a fluid. The channel can have any cross-sectional shape (circular, oval, triangular, irregular, square, rectangular, or the like) and can be covered or uncovered. In embodiments where it is covered, at least one portion of the channel can have a cross-section that is completely enclosed, or the entire channel may be completely enclosed along its entire length with the exception of its inlet(s) and outlet(s). A channel may also have an aspect ratio (length to average cross-sectional dimension) of at least about 3:1, at least about 5:1, or at least about 10:1 or more. An open channel generally will include characteristics that facilitate control over fluid transport, e.g., structural characteristics (an elongated indentation) and/or physical or chemical characteristics (hydrophobicity versus hydrophilicity) or other characteristics that can exert a force (e.g., a containing force) on a fluid. The fluid within the channel may partially or completely fill the channel. In some cases where an open channel is used, the fluid may be held within the channel, for example, using surface tension.

By “target analyte” is meant any atom, molecule, ion, molecular ion, compound, particle, cell, virus, complex, or fragment thereof to be either detected, measured, quantified, or evaluated. A target analyte may be contained in a sample (e.g., a liquid sample (e.g., a biological sample or an environmental sample)). Exemplary target analytes include, without limitation, a small molecule (e.g., an organic compound, a steroid, a hormone, a hapten, a biogenic amine, an antibiotic, a mycotoxin, an organic pollutant, a nucleotide, an amino acid, a monosaccharide, or a secondary metabolite), a protein (including a glycoprotein or a prion), a nucleic acid (e.g., a modified nucleic acid or an miRNA), a polysaccharide, a lipid, an extracellular vesicle, a glycan, a toxin, a fatty acid, a cell, a gas, a therapeutic agent, an organism (e.g., a pathogen), or a virus. The target analyte may be naturally occurring or synthetic. In some embodiments, a target analyte is an interferon, e.g., interferon γ (IFNγ). In some embodiments, a target analyte is an interleukin, e.g., interleukin 2 (IL-2).

The terms “nucleic acid” and “polynucleotide,” as used interchangeably herein, refer to at least two covalently linked nucleotide monomers. The term encompasses, e.g., deoxyribonucleic acid (DNA), ribonucleic acid (RNA), hybrids thereof, and mixtures thereof. Nucleotides are typically linked in a nucleic acid by phosphodiester bonds, although the term “nucleic acid” also encompasses nucleic acid analogs having other types of linkages or backbones (e.g., phosphorothioate, phosphoramide, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, and the like). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases.

By “protein” herein is meant at least two covalently linked amino acids, which includes proteins, polypeptides, oligopeptides and peptides. The protein may be made up of naturally occurring amino acids and peptide bonds, or synthetic peptidomimetic structures. Thus “amino acid,” or “peptide residue,” as used herein, means both naturally occurring and synthetic amino acids. For example, homo-phenylalanine, citrulline and norleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. The term “portion” includes any region of a protein, such as a fragment (e.g., a cleavage product or a recombinantly-produced fragment) or an element or domain (e.g., a region of a polypeptide having an activity) that contains fewer amino acids than the full-length or reference polypeptide (e.g., about 5%, 10%, 15%, 20%, 30%, 40%, 50%, 60%, 70%, 80%, 90%, 95%, or 99% fewer amino acids).

The term “small molecule,” as used herein, means any molecule having a molecular weight of less than 5000 Da. For example, in some embodiments, a small molecule is an organic compound, a steroid, a hormone, a hapten, a biogenic amine, an antibiotic, a mycotoxin, a cyanotoxin, a nitro compound, a drug residue, a pesticide residue, an organic pollutant, a nucleotide, an amino acid, a monosaccharide, or a secondary metabolite.

The term “capture probe,” as used herein, means a moiety to which a target analyte can be conjugated, captured, attached, bound, or affixed. In some embodiments, a target analyte is conjugated, captured, attached, bound, or affixed to a capture probe by a capture ligand. Detection probes or detectable moieties may bind or otherwise associate with a capture probe in single molecule array assays as described herein. Suitable capture probes include, but are not limited to, beads (e.g., magnetic beads (e.g., paramagnetic beads), silica beads, or hydrogel beads), nanotubes, polymers, or the like. In some embodiments, a droplet holds zero or one capture probes. In other embodiments, a droplet may hold more than one capture probe.

The term “capture ligand,” as used herein, means a moiety that is capable of specifically binding to or otherwise specifically associating with a capture probe or a target analyte. A capture ligand may be conjugated, captured, attached, bound, or affixed to a capture probe. For example, in some embodiments, a capture ligand is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), an antibody IgG binding protein (e.g., protein A, protein G, protein L, or recombinant protein A/G), a polypeptide, a nucleic acid, or a small molecule. For example, in some embodiments, a capture ligand binds to an Fc region of an antibody.

The terms “bead,” “particle,” and “microsphere,” as used interchangeably herein, mean a small discrete particle. Suitable beads include, but are not limited to, magnetic beads (e.g., paramagnetic beads), plastic beads, ceramic beads, glass beads, silica beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In some embodiments, spherical beads are used, but non-spherical or irregularly-shaped beads may be used.

The term “detection probe,” as used herein, means any molecule, particle, or the like that is capable of specifically binding to or otherwise specifically associating with a target analyte or another molecule that binds to or otherwise associates with the target analyte (e.g., another detection probe). For example, in some embodiments, a detection probe is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule.

The term “detectable moiety,” as used herein, means a moiety that can produce a detectable signal. For example, in some embodiments, a detectable moiety is or comprises an enzymatic label (e.g., beta-galactosidase, horseradish peroxidase, glucose oxidase, and alkaline phosphatase), a fluorescent label, a radioactive label, or a metal label. In particular embodiments, the detectable moiety is beta-galactosidase.

The term “immobilized target analyte,” as used herein, means a target analyte that is conjugated, captured, attached, bound, or affixed to a composition (e.g., a capture probe or a detectable moiety) to prevent or minimize dissociation or loss of the target analyte, but does not require absolute immobility with respect to the composition (e.g., the capture probe or the detectable moiety). The target analyte may be covalently or non-covalently immobilized, e.g., to a capture probe or a detectable moiety. In several embodiments, immobilized target analytes are used in competitive immunoassays as described herein, for example, and may compete with target analytes contained in a sample (e.g., a biological or environmental sample) for binding to a detection probe (e.g., an antibody).

The term “non-covalent affinity binding pair” refers to a pair of moieties that bind and form a non-covalent complex. Exemplary non-covalent affinity binding pairs include, without limitation, biotin-biotin binding protein (e.g., biotin-streptavidin and biotin-avidin), ligand-receptor, antigen-antibody or antigen binding fragment, hapten-anti-hapten, and immunoglobulin (Ig) binding protein-Ig. The members of a non-covalent affinity binding pair may have any suitable binding affinity. For example, the members of an affinity binding pair may bind with an equilibrium dissociation constant (K_{D} or Kd) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or lower.

A “pathogen” is an agent that can cause a disease or illness to its host, including, without limitation, a virus (e.g., a parvovirus (e.g., an adeno-associated virus (AAV)), a retrovirus (e.g., a lentivirus (e.g., human immunodeficiency virus (HIV))), a herpesvirus, an adenovirus, and the like), a bacterium (e.g., E. coli), a protozoon, a fungus, or a prion.

As used herein, “subject” means any animal. In one embodiment, the subject is a human. Other animals that can be subjects include but are not limited to non-human primates (e.g., monkeys, gorillas, and chimpanzees), domesticated animals (e.g., horses, pigs, donkeys, goats, rabbits, sheep, cattle, yaks, alpacas, and llamas), and companion animals (e.g., cats, lizards, snakes, dogs, fish, hamsters, guinea pigs, rats, mice, and birds).

As used herein, “biomarker” and “marker” interchangeably refer to an analyte (e.g., a small molecule, DNA, RNA, protein, carbohydrate, or glycolipid-based molecular marker), the expression or presence of which in a subject's sample can be detected by methods described herein and is useful, for example, for determining a prognosis, or for monitoring the responsiveness or sensitivity of a subject to a therapeutic agent.

The term “liquid sample,” as used herein, means a sample that is substantially in liquid form. A liquid sample may include, for example, a biological sample or an environmental sample. It is to be understood that a liquid sample may contain, e.g., particulates or other solid matter. In some embodiment, the liquid sample is a serum sample.

As used herein, “biological sample” refers to any biological sample obtained from or derived from a subject, including body fluids, body tissue (e.g., tumor tissue), cells, or other sources. Body fluids are, e.g., lymph, whole blood (including fresh or frozen), plasma (including fresh or frozen), serum (including fresh or frozen), a blood fraction containing peripheral blood mononuclear cells, urine, saliva, semen, sweat, lacrimal fluid, synovial fluid, cerebrospinal fluid, feces, mucous, vaginal fluid, and spinal fluid. Samples also include breast tissue, renal tissue, colonic tissue, brain tissue, muscle tissue, synovial tissue, skin, hair follicle, bone marrow, tumor tissue, a tissue lysate or homogenate, or an organ lysate or homogenate. Methods for obtaining tissue biopsies and body fluids from mammals are well known in the art.

By “environmental sample” is meant any sample that is obtained from an environment, e.g., a water sample, soil sample, air sample, extraterrestrial materials, and the like. An environmental sample may contain biological molecules or organisms.

A first moiety “specifically binds” (or grammatical variants thereof) a second moiety if the first moiety (e.g., a detection probe) binds to the second moiety (e.g., a target analyte or an immobilized target analyte) with specificity sufficient to differentiate between the second moiety and other components or contaminants of the test sample. The binding is generally sufficient to remain bound under the conditions of the assay, including wash steps to remove non-specific binding, although in some embodiments, wash steps are not desired; i.e., for detecting low affinity binding partners. In some embodiments, a first moiety specifically binds to a second moiety with an equilibrium dissociation constant (K_{D}) of about 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, 10⁻¹⁵ M, or lower.

### II. Methods

The invention provides methods of detecting a target analyte in a liquid sample, e.g., a serum sample. The methods can also involve measuring a concentration or amount of a target analyte, e.g., a protein molecule. Detection may be direct or indirect, as described further below. In several embodiments, the target analyte is introduced into a plurality of droplets, and single molecules can be detected in the droplets.

Detection of the target analyte may be direct. For example, provided herein is a method of detecting a target analyte in a sample that includes one, two, three, or all four of the following steps: (a) contacting a sample containing or suspected of containing a target analyte with a plurality of capture probes, the capture probes being linked to one or more capture ligands that specifically bind to the target analyte, and incubating to allow binding of the capture ligands to the target analytes; (b) contacting the product of (a) with a plurality of detection probes that specifically bind to the target analyte, and incubating to allow binding of the detection probes to the target analyte, the detection probes each being linked to a detectable moiety; (c) producing a plurality of droplets of the product of step (b); and (d) detecting the detectable moieties present in the plurality of droplets, thereby detecting the target analyte in the sample. The sample may be contacted with the capture probes and the detection probes sequentially or simultaneously. In some embodiments, all or substantially all of the droplets contain zero or one capture probes. In other embodiments, some, all, or substantially all of the droplets contain more than one (e.g., about two, three, four, five, six, seven, eight, nine, ten, or more) capture probes.

In other embodiments, an indirect detection approach can be used. Any suitable indirect detection approach (e.g., competitive binding) can be used. For example, in some embodiments, the method may include one, two, or three of the following steps: (a) contacting a sample containing or suspected of containing a target analyte with a plurality of capture probes, the capture probes being reversibly linked to one or more detection probes that specifically bind to the target analyte, and incubating to allow binding of the detection probes to the target analyte, the detection probes each being linked to a detectable moiety; (b) producing a plurality of droplets from the product of step (a); and (c) detecting the detectable moieties present in the plurality of droplets, thereby detecting the target analyte in the sample. In such method, binding of the detection probes to the target analyte in the sample will reduce the number of detection probes that are linked to the capture probes, such that the signal of the detectable moieties is inversely proportional to the amount of the target analyte in the sample.

In yet another embodiment, the invention provides a method of detecting a target analyte in a sample, the method including the steps of: (a) contacting a sample containing or suspected of containing a target analyte with: (i) a plurality of detection probes that specifically bind to the target analyte, the detection probes being linked to a detectable moiety, and (ii) a plurality of capture probes, the capture probes being linked to one or more immobilized target analytes, wherein the detection probes competitively bind to the target analytes contained in the sample and to the immobilized target analytes; (b) incubating the product of step (a) to allow binding of the detection probes to the target analytes contained in the sample or to the immobilized target analytes; (c) producing a plurality of droplets of the product of step (b); and (d) detecting the detectable moieties in the droplets, thereby detecting the target analyte in the sample. In such methods, the concentration of the target analyte in the sample is inversely proportional to the signal of the detectable moieties. In some embodiments, all or substantially all of the capture probes of step (b) are associated with either zero or one detection probe, wherein a detection probe is associated with a capture probe by binding to a linked immobilized target analyte.

In a further embodiment, the invention provides a method of detecting a target analyte in a sample, the method including the steps of: (a) contacting a sample containing or suspected of containing a target analyte with: (i) a plurality of detection probes that specifically bind to the target analyte, and (ii) a plurality of capture probes, the capture probes being linked to one or more immobilized target analytes, wherein the detection probes competitively bind to the target analytes contained in the sample and to the immobilized target analytes; (b) incubating the product of step (a) to allow binding of the detection probes to the target analytes contained in the sample or to the immobilized target analytes; (c) labeling the detection probes that are bound to the immobilized target analytes linked to the capture probes of step (b) with detectable moieties; (d) producing a plurality of droplets of the product of step (c); and (e) detecting the detectable moieties in the droplets, thereby detecting the target analyte in the sample. In such methods, the concentration of the target analyte in the sample is inversely proportional to the signal of the detectable moieties. In some embodiments, all or substantially all of the capture probes of step (c) are associated with either zero or one detection probe, wherein a detection probe is associated with a capture probe by binding to a linked immobilized target analyte.

In a still further aspect, the invention provides a method of detecting a target analyte in a sample, the method including the steps of: (a) contacting a sample containing or suspected of containing a target analyte with: (i) a plurality of detection probes that specifically bind to the target analyte, and (ii) a plurality of detectable moieties, the detectable moieties being linked to one or more immobilized target analytes, wherein the detection probes competitively bind to the target analytes contained in the sample and to the immobilized target analytes; (b) incubating the product of step (a) to allow binding of the detection probes to the target analytes contained in the sample or to the immobilized target analytes; (c) contacting the product of step (b) with a plurality of capture probes, the capture probes being linked to one or more capture ligands, wherein the capture ligand specifically binds to the detection probe, and incubating to allow capture ligands to bind to detection probes; (d) producing a plurality of droplets of the product of step (c); and (e) detecting the detectable moieties that are associated with the capture probes of step (d), wherein detectable moieties are associated with capture probes by binding of a linked immobilized target analyte to a detection probe that is bound to a capture ligand linked to the capture probe, thereby detecting the target analyte in the sample. In such methods, the concentration of the target analyte in the sample is inversely proportional to the signal of the detectable moieties. In some embodiments, all or substantially all of the capture probes of step (d) are associated with either zero or one detectable moiety.

Some, all, or substantially all of the droplets can contain zero or one capture probes. In other embodiments, some, all, or substantially all of the droplets can contain more than one (e.g., about two, three, four, five, six, seven, eight, nine, ten, or more) capture probes.

The methods can be multiplexed for detection of more than one target analyte, e.g., two, three, four, five, six, seven, eight, nine, ten, twenty, or more target analytes. For example, provided herein is a method of detecting a first target analyte and a second target analyte in a sample, the method including one, two, three, or all four of the following steps: (a) contacting a sample containing or suspected of containing a first target analyte and a second target analyte with: a plurality of first capture probes, the first capture probes being linked to one or more first capture ligands that specifically bind to the first target analyte; and a plurality of second capture probes, the second capture probes being linked to one or more second capture ligands that specifically bind to the second target analyte, and incubating to allow binding of the first and second capture ligands to the first and second target analytes, respectively; (b) contacting the product of (a) with: a plurality of first detection probes that specifically bind to the first target analyte, and a plurality of second detection probes that specifically bind to the second target analyte, and incubating to allow binding of the first and second detection probes to the first and second target analytes, respectively, wherein the first and second detection probes are each labelled with a detectable moiety; (c) producing a plurality of droplets from the product of (b); and (d) detecting the detectable moieties present in the plurality of droplets, thereby detecting the first target analyte and the second target analyte in the sample.

In such multiplexed methods, either or both of the capture probes or the detectable moieties can be distinguishably labelled. For example, in some embodiments, the first capture probe and the second capture probe are detectably and distinguishably labeled, and step (d) comprises detecting the capture probes and the detectable moieties present in the plurality of droplets. Any suitable number of dyes, as well as any suitable dye intensities, may be used to generate distinguishable capture probes. For example, in some embodiments, the first capture probe may be labelled with one or more dyes (e.g., one, two, three, four, five, six, seven, eight, nine, or ten dyes), and the second capture probe may be labelled with one or more dyes (e.g., one, two, three, four, five, six, seven, eight, nine, or ten dyes), such that the first and second capture probes are distinguishably labeled. In some embodiments, the first capture probe is labelled with a first dye, and the second capture probe is labelled with a second dye. In some embodiments, the first detection probe is labelled with a first detectable moiety, and the second detection probe is labelled with a second detectable moiety, and the first detectable moiety and the second detectable moiety are distinguishable.

In some embodiments, the capture probes are linked to from about 1 to about 10¹² capture ligands or more, e.g., about 1, about 10, about 100, about 1000, about 10,000, about 100,000, about 1,000,000, about 10,000,000, about 100,000,000, about 10⁹, about 10¹⁰, about 10¹¹, or about 10¹² capture ligands.

The methods may be used to detect a target analyte having any suitable concentration in the liquid sample. For example, the concentration of the target analyte in the liquid sample can be in the attomolar (aM), femtomolar (fM), picomolar (pM), nanomolar (nM), micromolar (μM), or millimollar (mM) ranges. For example, the concentration of the target analyte in the liquid sample may be about 0 aM to about 10 mM, e.g., about 0 aM, about 10 aM, about 100 aM, about 1 fM, about 10 fM, about 100 fM, about 1 pM, about 10 pM, about 100 pM, about 1 nM, about 10 nM, about 100 nM, about 1 μM, about 10 μM, about 100 μM, about 1 mM, or about 10 mM. In some embodiments, the concentration of the target analyte in the liquid sample may be about 0 aM to about 10 aM, about 0 aM to about 100 aM, about 0 aM to about 1 fM, about 0 aM to about 10 fM, about 0 aM to about 100 fM, about 0 aM to about 1 pM, about 0 aM to about 10 pM, about 0 aM to about 100 pM, about 0 aM to about 1 nM, about 0 aM to about 10 nM, about 0 aM to about 100 nM, about 0 aM to about 1 μM, about 0 aM to about 10 μM, about 0 aM to about 100 μM, about 0 aM to about 1 mM, about 0 aM to about 10 mM, about 1 aM to about 10 aM, about 1 aM to about 100 aM, about 1 aM to about 1 fM, about 1 aM to about 10 fM, about 1 aM to about 100 fM, about 1 aM to about 1 pM, about 1 aM to about 10 pM, about 1 aM to about 100 pM, about 1 aM to about 1 nM, about 1 aM to about 10 nM, about 1 aM to about 100 nM, about 1 aM to about 1 μM, about 1 aM to about 10 μM, about 1 aM to about 100 μM, about 1 aM to about 1 mM, about 1 aM to about 10 mM, about 5 aM to about 10 aM, about 5 aM to about 100 aM, about 5 aM to about 1 fM, about 5 aM to about 10 fM, about 5 aM to about 100 fM, about 5 aM to about 1 pM, about 5 aM to about 10 pM, about 5 aM to about 100 pM, about 5 aM to about 1 nM, about 5 aM to about 10 nM, about 5 aM to about 100 nM, about 5 aM to about 1 μM, about 5 aM to about 10 μM, about 5 aM to about 100 μM, about 5 aM to about 1 mM, about 5 aM to about 10 mM, about 10 aM to about 100 aM, about 10 aM to about 1 fM, about 10 aM to about 10 fM, about 10 aM to about 100 fM, about 10 aM to about 1 pM, about 10 aM to about 10 pM, about 10 aM to about 100 pM, about 10 aM to about 1 nM, about 10 aM to about 10 nM, about 10 aM to about 100 nM, about 10 aM to about 1 μM, about 10 aM to about 10 μM, about 10 aM to about 100 μM, about 10 aM to about 1 mM, about 10 aM to about 10 mM, about 100 aM to about 1 fM, about 100 aM to about 10 fM, about 100 aM to about 100 fM, about 100 aM to about 1 pM, about 100 aM to about 10 pM, about 100 aM to about 100 pM, about 100 aM to about 1 nM, about 100 aM to about 10 nM, about 100 aM to about 100 nM, about 100 aM to about 1 μM, about 100 aM to about 10 μM, about 100 aM to about 100 μM, about 100 aM to about 1 mM, or about 100 aM to about 10 mM. In some embodiments, the concentration of the target analyte is about 1 aM to about 100 fM. In some embodiments, the concentration of target analyte is about 10 aM to about 100 fM. In some embodiments, the concentration of target analyte is about 100 aM to about 100 fM. In some embodiments, the concentration of target analyte is about 1 fM to about 100 fM. In some embodiments, the concentration of target analyte is about 10 fM to about 100 fM. In some embodiments, the concentration of target analyte is about 10 fM to about 100 fM.

Any suitable duration of incubating can be used in the methods described herein. The incubating can be performed for about 1 min to about 48 h, e.g., about 1 min, about 5 min, about 10 min, about 20 min, about 30 min, about 40 min, about 50 min, about 60 min, about 2 h, about 3 h, about 4 h, about 5 h, about 6 h, about 7 h, about 8 h, about 9 h, about 10 h, about 11 h, about 12 h, about 13 h, about 14 h, about 15 h, about 16 h, about 17 h, about 18 h, about 19 h, about 20 h, about 21 h, about 22 h, about 23 h, about 24 h, about 25 h, about 26 h, about 27 h, about 28 h, about 29 h, about 30 h, about 40 h, or about 48 h.

The droplets may have any suitable size or volume, for example, as described below in Section III (“Droplet Arrays”). In some embodiments, the droplets have a volume of about 0.001 pL to about 100 nL, e.g., about 0.01 pL to about 10 nL, about 0.01 pL to about 1 nL, about 0.01 pL to about 100 pL, about 0.01 pL to about 100 pL, about 0.1 pL to about 100 pL, or about 0.1 pL to about 10 pL.

Any suitable approach may be used for producing droplets, e.g., as described below in Section III (“Droplet Arrays”). In particular embodiments, the droplet production is performed using a microfluidic device, e.g., a flow-focusing device.

Any suitable target analyte can be detected and, optionally, quantified using the methods described herein. In some embodiments, the target analyte is any target analyte described herein (see, e.g., Section V, “Target Analytes”). In some embodiments, the target analyte is a biomarker.

Any suitable capture ligand or detection probe can be used in the invention. The capture ligand and/or the detection probe can be an antibody, an aptamer, an antibody mimetic, a polypeptide, a nucleic acid, a molecularly-imprinted polymer, a receptor, or a small molecule. The antibody may be a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb). The antibody mimetic may be wherein the antibody mimetic is an affibody, an affilin, an affimer, an affitin, an alphabody, an anticalin, an avimer, a DARPin, a fynomer, a Kunitz domain peptide, a monobody, or a nanoCLAMP. In some embodiments, the capture ligand and the detection probe are the same molecule, e.g., antibody. In some embodiments, the capture ligand and the detection probe specifically target the same epitope on the target analyte. In some embodiments, the capture ligand and the detection probe specifically target different epitopes on the target analyte. In some embodiments, the capture ligand and the detection probe are different molecules, e.g., antibodies.

Any suitable capture probe can be used in the context of the invention. The capture probe can be a bead (e.g., a magnetic bead (e.g., a paramagnetic bead), a silica bead, or a hydrogel bead), a nanotube, or a polymer. In particular embodiments, the capture probe is a magnetic bead (e.g., a paramagnetic bead). In some embodiments, the beads have a size (e.g., a diameter) of about 0.01 μm to about 100 μm, e.g., about 0.01 μm, about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 1.5 μm, about 2 μm, about 2.5 μm, about 3 μm, about 3.5 μm, about 4 μm, about 4.5 μm, about 6 μm, about 6.5 μm, about 7 μm, about 7.5 μm, about 8 μm, about 8.5 μm, about 9 μm, about 9.5 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 55 μm, about 60 μm, about 65 μm, about 70 μm, about 75 μm, about 80 μm, about 85 μm, about 90 μm, about 95 μm, or about 100 μm. In some embodiments, the beads have a size of about 1 μm to about 50 μm, about 1 μm to about 25 μm, about 1 μm to about 10 μm, about 1 μm to about 5 μm, about 1 μm to about 4 μm, about 1 μm to about 3 μm, or about 1 μm to about 2 μm. In particular embodiments, the beads have a size of about 1 μm to about 50 μm.

Any of the methods described herein may involve contacting the liquid sample with about 1,000 to about 100,000,000 capture probes, e.g., about 1000, about 10,000, about 20,000, about 30,000, about 40,000, about 50,000, about 60,000, about 70,000, about 80,000, about 90,000, about 100,000, about 200,000, about 300,000, about 400,000, about 500,000, about 600,000, about 700,000, about 800,000, about 900,000, about 1,000,000, about 2,000,000, about 3,000,000, about 4,000,000, about 5,000,000, about 10,000,000, about 50,000,000, or about 100,000,000 capture probes. In some embodiments, the method may involve contacting the liquid sample with about 10,000 to about 5,000,000 capture probes, about 10,000 to about 4,000,000 capture probes, about 10,000 to about 3,000,000 capture probes, about 10,000 to about 2,000,000 capture probes, about 10,000 to about 1,000,000 capture probes, about 10,000 to about 500,000 capture probes, about 10,000 to about 400,000 capture probes, about 10,000 to about 300,000 capture probes, about 10,000 to about 200,000 capture probes, or about 10,000 to about 100,000 capture probes.

The detectable moiety can be or can include an enzymatic label (e.g., beta-galactosidase, horseradish peroxidase, glucose oxidase, and alkaline phosphatase), a fluorescent label, a radioactive label, or a metal label. In some embodiments, droplet production includes mixing the liquid sample and the capture probes with an enzyme substrate. The enzyme substrate can be pre-mixed with the capture probes before droplet production. For example, in some embodiments, an enzymatic label generates a species (for example, a fluorescent product) that is either directly or indirectly detectable optically. In some embodiments, the method includes detecting a product of an enzymatic reaction as an indication of the presence of the enzymatic label. In some embodiments, the product of the enzymatic reaction is detected upon its release from the enzymatic label in a zone around the discrete site where the enzyme and/or target analyte is located (e.g., in a droplet, for example, in an array of droplets, as described herein). In some embodiments, the enzyme substrate is fluorescein di-β-D-galactopyranoside (FDG). In some embodiments, the enzyme substrate is resorufin β-D-galactopyranoside (RGP). In some embodiments, the enzyme substrate is a horseradish peroxide (HRP) substrate. In some embodiments, the enzyme substrate is an alkaline phosphate substrate.

Any of the methods described herein may include mixing the contents of the droplets, for example, by chaotic advection using channels with turns in a microfluidic device. Additional approaches for mixing the droplets are described, for example, in Song et al. Angew. Chem. Int. Ed. 45:7336-7356, 2006 and Sarrazin et al. Chem. Eng. Sci. 62(4):1042-1048, 2007.

Any suitable liquid sample may be used in any of the methods described herein. In some embodiments, the liquid sample is or includes a biological sample or an environmental sample. Any suitable biological samples or environmental samples, or derivatives thereof, can be used in the preceding methods, including those described herein.

The methods may involve use of a density gradient medium. The capture probes may be mixed with a density gradient medium, which can be used to promote even distribution of the beads in solution and reduce or prevent aggregation of capture probes (e.g., beads). Use of a density gradient medium can facilitate isolation of one capture probe (e.g., a bead) in a droplet. Any suitable density gradient medium can be used, e.g., iodixanol solution (e.g., OPTIPREP™ iodixanol solution), polysaccharide (e.g., sucrose) polymers (e.g., FICOLL® (e.g., FICOLL®PM 400, FICOLL®PM 70, or FICOLL®-Paque), or colloidal media, for example, containing silica particles covalently coated with silane (e.g., PERCOLL® and PERCOLL® PLUS), NycoPrep™, NYCODENZ®, LymphoPrep™, PolymorphPrep, AXIS-SHIELD™, and the like. In some embodiments, the droplets contain a density gradient medium.

For example, in some cases, a measure of the concentration may be based at least in part on the number of droplets determined to contain a capture probe that is or was associated with at least one detectable moiety. In some embodiments, the number of droplets determined to contain a capture probe that is or was associated with at least one detectable moiety may be proportional to the concentration of the target analyte in the sample. In other embodiments, such as competitive immunoassays for detection of small molecules, the number of droplets determined to contain a capture probe that is or was associated with at least one detectable moiety may be inversely related to the concentration of the target analyte in the sample. In other cases and/or under differing conditions, a measure of the concentration may be based at least in part on an intensity level of at least one signal indicative of the presence of a plurality of target analyte molecules and/or capture probes associated with a target analyte molecule at one or more of the addressed locations. In some embodiments, the number/fraction of droplets containing a capture probe but not containing a detectable moiety or a target analyte may also be determined and/or the number/fraction of droplets not containing any capture probe may also be determined. In some embodiments, there are multiple capture probes (e.g., beads) per droplet. Since there is typically only one target analyte molecule bound per bead, and most beads do not have any bound target analyte molecule, single molecule detection can be achieved with multiple capture probes per droplet.

A statistically significant fraction of capture probes that contain at least one detectable moiety or target analyte (or no detectable moieties or target analytes) will typically be able to be reproducibly detected and quantified using a particular system of detection and will typically be above the background noise (e.g., non-specific binding) that is determined when carrying out the assay with a sample that does not contain any target analytes, divided by the total number of droplets addressed.

The total number of capture probes may be between about 10,000 and about 10,000,000,000, between about 50,000 and about 5,000,000, or between about 100,000 and about 1,000,000. In some cases, the total number of capture probes provided is at least about 10,000, at least about 50,000, at least about 100,000, at least about 1,000,000, at least about 5,000,000, at least about 10,000,000, at least about 100,000,000, at least about 200,000,000, at least about 300,000,000, at least about 400,000,000, at least about 500,000,000, at least about 600,000,000, at least about 700,000,000, at least about 800,000,000, at least about 900,000,000, at least about 1,000,000,000, at least about 2,000,000,000, at least about 3,000,000,000, at least about 4,000,000,000, or at least about 5,000,000,000. In some embodiments, the total number of capture probes for one assay is about 50,000. In some embodiments, the total number of capture probes for one assay is about 60,000. In some embodiments, the total number of capture probes for one assay is about 70,000. In some embodiments, the total number of capture probes for one assay is about 80,000. In some embodiments, the total number of capture probes for one assay is about 90,000. In some embodiments, the total number of capture probes for one assay is about 100,000.

A variety of other reagents may be included in the methods described herein. These include reagents like salts, neutral proteins, e.g., albumin, detergents, surfactants, density gradient media, and the like, which may be used to facilitate optimal protein-protein binding and/or reduce non-specific or background interactions. Reagents such as protease inhibitors, nuclease inhibitors, anti-microbial agents, etc., may be used. The mixture of components may be added in any order that provides for the requisite binding. Various blocking and washing steps may be utilized as is known in the art. For example, any of the preceding methods may include one or more (e.g., 1, 2, 3, 4, 5, 6, 7, 8, 9, or 10) wash steps.

Any of the methods described herein may involve providing a prognosis or a diagnosis for a subject based on the concentration of the one or more target analyte(s) in the sample. Any of the methods described herein may involve selecting a therapy for a patient based on the concentration of the one or more target analyte(s) in the sample. Any of the methods described herein may involve treating a subject with a therapy based on the concentration of the one or more target analyte(s) in the sample.

### III. Droplets and Droplet Arrays

In some embodiments, the methods described herein may utilize a plurality, e.g., in an array, of droplets to determine the presence or concentration of one or more target analytes. The plurality of droplets allows a fluid sample to be partitioned into a plurality of discrete reaction volumes during one or more steps of a method.

Any suitable approach may be used to produce the droplets used in the context of the invention. For example, the droplets may be formed by shaking or stirring a liquid to form individual droplets, creating a suspension or an emulsion containing individual droplets, or forming the droplets through pipetting techniques, needles, or the like. In particular embodiments, the plurality of droplets may be made using a micro-, or nanofluidic droplet maker, e.g., a T-junction droplet maker, a Y-junction droplet maker, a channel-within-a-channel junction droplet maker, a cross (or “X”) junction droplet maker, a flow-focusing junction droplet maker, a micro-capillary droplet maker (e.g., co-flow or flow-focus), a three-dimensional droplet maker, and the like. In particular embodiments, the droplets are produced using a flow-focusing device, for example, as described in Example 1. In other embodiments, a plurality of droplets may be formed using emulsification systems, for example, homogenization, membrane emulsification, shear cell emulsification, fluidic emulsification, and the like. Other non-limiting examples of the creation of droplets are disclosed in Mazutis et al. Nat. Protoc. 8(5):870-891, 2013; U.S. Pat. No. 9,839,911; U.S. Patent Application Publication Nos. 2005/0172476; 2006/0163385; and 2007/0003442; and in International Patent Application Publication Nos. WO 2009/005680 and WO 2018/009766. In some embodiments, electric fields or acoustic waves may be used to produce droplets, e.g., as described in WO 2018/009766. Other approaches for droplet production are known in the art. The device may contain a mixing unit, e.g., one or more channels with bends (e.g., 45° angle bends), winding channels, bumpy mixers, or the like, to promote chaotic advection of the droplets. See, e.g., Song et al. Angew. Chem. Int. Ed. 45:7336-7356, 2006 and Sarrazin et al. Chem. Eng. Sci. 62(4):1042-1048, 2007.

The device may contain any suitable number of channels or inlets, e.g., 1, 2, 3, 4, 5, 6, 7, 8, 9, 10, 15, 20, 25, 30, 40, 50, 60, 70, 80, 90, 100, or more channels or inlets. For example, in some embodiments, the device includes a first inlet for beads (which may include any additional assay reagents, such as an enzyme substrate) and a second inlet for oil. In other embodiments, the device may include a first inlet for beads, a second inlet for an enzyme substrate, and a third inlet for oil.

The droplets may be polydisperse, monodisperse, or substantially monodisperse (e.g., having a homogenous distribution of diameters). A plurality of droplets is substantially monodisperse in instances where the droplets have a distribution of diameters such that no more than about 10%, about 5%, about 4%, about 3%, about 2%, about 1%, or less, of the droplets have a diameter greater than or less than about 20%, about 30%, about 50%, about 75%, about 80%, about 90%, about 95%, about 99%, or more, of the average diameter of all of the droplets.

The average diameter of a population of droplets may be the arithmetic average of the diameters of the droplets. For example, in some embodiments, the droplets may have a diameter of from about 500 nm to about 1 mm, e.g., about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 100 μm, or greater. In some embodiments, the droplets may have a diameter of about 1 μm to about 100 μm, about 1 μm to about 90 μm, about 1 μm to about 80 μm, about 1 μm to about 70 μm, about 1 μm to about 60 μm, about 1 μm to about 50 μm, about 1 μm to about 40 μm, about 1 μm to about 30 μm, about 1 μm to about 20 μm, about 1 μm to about 10 μm, about 5 μm to about 100 μm, about 5 μm to about 90 μm, about 5 μm to about 80 μm, about 5 μm to about 70 μm, about 5 μm to about 60 μm, about 5 μm to about 50 μm, about 5 μm to about 40 μm, about 5 μm to about 30 μm, about 5 μm to about 20 μm, about 5 μm to about 10 μm, about 10 μm to about 100 μm, about 10 μm to about 90 μm, about 10 μm to about 80 μm, about 10 μm to about 70 μm, about 10 μm to about 60 μm, about 10 μm to about 50 μm, about 10 μm to about 40 μm, about 10 μm to about 30 μm, about 10 μm to about 20 μm, about 20 μm to about 100 μm, about 20 μm to about 90 μm, about 20 μm to about 80 μm, about 20 μm to about 70 μm, about 20 μm to about 60 μm, about 20 μm to about 50 μm, about 20 μm to about 40 μm, or about 20 μm to about 30 μm.

The droplets may have any suitable volume. In some embodiments, the droplets may all have approximately the same volume. In other embodiments, the droplets may have different volumes. The volume of each individual droplets can range, for example, from attoliters or smaller to nanoliters or larger depending upon the nature of analyte molecules, the detection technique and equipment employed, and the expected concentration of the analyte molecules in the fluid applied to the array for analysis. The size of the droplets may be selected such that at the concentration of interest, between zero and ten capture probes would be statistically expected to be found in each droplet. In a particular embodiment, the volume of the droplet is selected such that at the concentration of interest, either zero or one capture probes would be statistically expected to be found in each reaction vessel.

For example, in some embodiments, the droplets may have a volume of about 1 aL to about 100 nL, e.g., about 0.01 pL to about 10 nL. In other embodiments, the droplets may have a volume between about 1 femtoliter and about 1 picoliter, between about 10 femtoliters and about 100 femtoliters, between about 10 attoliters and about 50 picoliters, between about 1 picoliter and about 50 picoliters, between about 1 picoliter and about 500 picoliters, between about 1 femtoliter and about 1 picoliter, between about 30 femtoliters and about 60 femtoliters, or the like. In some embodiments, the droplets have a volume of less than about 1 picoliter, less than about 500 femtoliters, less than about 100 femtoliters, less than about 50 femtoliters, less than about 1 femtoliter, or the like. In some embodiments, the droplets have a volume of about 10 femtoliters, about 20 femtoliters, about 30 femtoliters, about 40 femtoliters, about 50 femtoliters, about 60 femtoliters, about 70 femtoliters, about 80 femtoliters, about 90 femtoliters, or of about 100 femtoliters.

For example, in some embodiments, the droplets may have a volume of about 0.001 pL, about 0.01 pL, about 1 pL, about 2 pL, about 5 pL, about 10 pL, about 15 pL, about 20 pL, about 25 pL, about 30 pL, about 40 pL, about 50 pL, about 100 pL, about 200 pL, about 300 pL, about 400 pL, about 500 pL, about 600 pL, about 700 pL, about 800 pL, about 900 pL, about 1 nL, about 2 nL, about 3 nL, about 4 nL, about 5 nL, about 6 nL, about 7 nL, about 8 nL, about 9 nL, about 10 nL, about 20 nL, about 30 nL, about 40 nL, about 50 nL, about 60 nL, about 70 nL, about 80 nL, about 90 nL, about 100 nL or greater. In some embodiments, the droplets may have a volume of about 1 pL to about 10 pL, about 1 pL to about 20 pL, about 1 pL to about 25 pL, about 1 pL to about 30 pL, about 1 pL to about 35 pL, about 1 pL to about 40 pL, about 1 pL to about 50 pL, about 1 pL to about 55 pL, about 1 pL to about 60 pL, about 1 pL to about 65 pL, about 1 pL to about 70 pL, about 1 pL to about 75 pL, about 1 pL to about 80 pL, about 1 pL to about 85 pL, about 1 pL to about 90 pL, about 1 pL to about 95 pL, about 1 pL to about 100 pL, about 1 pL to about 200 pL, about 1 pL to about 300 pL, about 1 pL to about 400 pL, about 1 pL to about 500 pL, about 1 pL to about 600 pL, about 1 pL to about 700 pL, about 1 pL to about 800 pL, about 1 pL to about 900 pL, about 1 pL to about 1 nL, about 1 pL to about 2 nL, about 1 pL to about 3 nL, about 1 pL to about 4 nL, about 1 pL to about 5 nL, about 1 pL to about 6 nL, about 1 pL to about 7 nL, about 1 pL to about 8 nL, about 1 pL to about 9 nL, about 1 pL to about 10 nL, about 5 pL to about 10 pL, about 5 pL to about 20 pL, about 5 pL to about 25 pL, about 5 pL to about 30 pL, about 5 pL to about 35 pL, about 5 pL to about 40 pL, about 5 pL to about 50 pL, about 5 pL to about 55 pL, about 5 pL to about 60 pL, about 5 pL to about 65 pL, about 5 pL to about 70 pL, about 5 pL to about 75 pL, about 5 pL to about 80 pL, about 5 pL to about 85 pL, about 5 pL to about 90 pL, about 5 pL to about 95 pL, about 5 pL to about 100 pL, about 5 pL to about 200 pL, about 5 pL to about 300 pL, about 5 pL to about 400 pL, about 5 pL to about 500 pL, about 5 pL to about 600 pL, about 5 pL to about 700 pL, about 5 pL to about 800 pL, about 5 pL to about 900 pL, about 5 pL to about 1 nL, about 5 pL to about 2 nL, about 5 pL to about 3 nL, about 5 pL to about 4 nL, about 5 pL to about 5 nL, about 5 pL to about 6 nL, about 5 pL to about 7 nL, about 5 pL to about 8 nL, about 5 pL to about 9 nL, about 5 pL to about 10 nL, about 10 pL to about 20 pL, about 10 pL to about 25 pL, about 10 pL to about 30 pL, about 10 pL to about 35 pL, about 10 pL to about 40 pL, about 10 pL to about 50 pL, about 10 pL to about 55 pL, about 10 pL to about 60 pL, about 10 pL to about 65 pL, about 10 pL to about 70 pL, about 10 pL to about 75 pL, about 10 pL to about 80 pL, about 10 pL to about 85 pL, about 10 pL to about 90 pL, about 10 pL to about 95 pL, about 10 pL to about 100 pL, about 10 pL to about 200 pL, about 10 pL to about 300 pL, about 10 pL to about 400 pL, about 10 pL to about 500 pL, about 10 pL to about 600 pL, about 10 pL to about 700 pL, about 10 pL to about 800 pL, about 10 pL to about 900 pL, about 10 pL to about 1 nL, about 10 pL to about 2 nL, about 10 pL to about 3 nL, about 10 pL to about 4 nL, about 10 pL to about 5 nL, about 10 pL to about 6 nL, about 10 pL to about 7 nL, about 10 pL to about 8 nL, about 10 pL to about 9 nL, about 10 pL to about 10 nL, about 20 pL to about 25 pL, about 20 pL to about 30 pL, about 20 pL to about 35 pL, about 20 pL to about 40 pL, about 20 pL to about 50 pL, about 20 pL to about 55 pL, about 20 pL to about 60 pL, about 20 pL to about 65 pL, about 20 pL to about 70 pL, about 20 pL to about 75 pL, about 20 pL to about 80 pL, about 20 pL to about 85 pL, about 20 pL to about 90 pL, about 20 pL to about 95 pL, about 20 pL to about 100 pL, about 20 pL to about 200 pL, about 20 pL to about 300 pL, about 20 pL to about 400 pL, about 20 pL to about 500 pL, about 20 pL to about 600 pL, about 20 pL to about 700 pL, about 20 pL to about 800 pL, about 20 pL to about 900 pL, about 20 pL to about 1 nL, about 20 pL to about 2 nL, about 20 pL to about 3 nL, about 20 pL to about 4 nL, about 20 pL to about 5 nL, about 20 pL to about 6 nL, about 20 pL to about 7 nL, about 20 pL to about 8 nL, about 20 pL to about 9 nL, about 20 pL to about 10 nL, about 30 pL to about 35 pL, about 30 pL to about 40 pL, about 30 pL to about 50 pL, about 30 pL to about 55 pL, about 30 pL to about 60 pL, about 30 pL to about 65 pL, about 30 pL to about 70 pL, about 30 pL to about 75 pL, about 30 pL to about 80 pL, about 30 pL to about 85 pL, about 30 pL to about 90 pL, about 30 pL to about 95 pL, about 30 pL to about 100 pL, about 30 pL to about 200 pL, about 30 pL to about 300 pL, about 30 pL to about 400 pL, about 30 pL to about 500 pL, about 30 pL to about 600 pL, about 30 pL to about 700 pL, about 30 pL to about 800 pL, about 30 pL to about 900 pL, about 30 pL to about 1 nL, about 30 pL to about 2 nL, about 30 pL to about 3 nL, about 30 pL to about 4 nL, about 30 pL to about 5 nL, about 30 pL to about 6 nL, about 30 pL to about 7 nL, about 30 pL to about 8 nL, about 30 pL to about 9 nL, about 30 pL to about 10 nL, about 40 pL to about 50 pL, about 40 pL to about 55 pL, about 40 pL to about 60 pL, about 40 pL to about 65 pL, about 40 pL to about 70 pL, about 40 pL to about 75 pL, about 40 pL to about 80 pL, about 40 pL to about 85 pL, about 40 pL to about 90 pL, about 40 pL to about 95 pL, about 40 pL to about 100 pL, about 40 pL to about 200 pL, about 40 pL to about 300 pL, about 40 pL to about 400 pL, about 40 pL to about 500 pL, about 40 pL to about 600 pL, about 40 pL to about 700 pL, about 40 pL to about 800 pL, about 40 pL to about 900 pL, about 40 pL to about 1 nL, about 40 pL to about 2 nL, about 40 pL to about 3 nL, about 40 pL to about 4 nL, about 40 pL to about 5 nL, about 40 pL to about 6 nL, about 40 pL to about 7 nL, about 40 pL to about 8 nL, about 40 pL to about 9 nL, about 40 pL to about 10 nL, about 50 pL to about 55 pL, about 50 pL to about 60 pL, about 50 pL to about 65 pL, about 50 pL to about 70 pL, about 50 pL to about 75 pL, about 50 pL to about 80 pL, about 50 pL to about 85 pL, about 50 pL to about 90 pL, about 50 pL to about 95 pL, about 50 pL to about 100 pL, about 50 pL to about 200 pL, about 50 pL to about 300 pL, about 50 pL to about 400 pL, about 50 pL to about 500 pL, about 50 pL to about 600 pL, about 50 pL to about 700 pL, about 50 pL to about 800 pL, about 50 pL to about 900 pL, about 50 pL to about 1 nL, about 50 pL to about 2 nL, about 50 pL to about 3 nL, about 50 pL to about 4 nL, about 50 pL to about 5 nL, about 50 pL to about 6 nL, about 50 pL to about 7 nL, about 50 pL to about 8 nL, about 50 pL to about 9 nL, about 50 pL to about 10 nL, about 100 pL to about 600 pL, about 100 pL to about 700 pL, about 100 pL to about 800 pL, about 100 pL to about 900 pL, about 100 pL to about 1 nL, about 100 pL to about 2 nL, about 100 pL to about 3 nL, about 100 pL to about 4 nL, about 100 pL to about 5 nL, about 100 pL to about 6 nL, about 100 pL to about 7 nL, about 100 pL to about 8 nL, about 100 pL to about 9 nL, about 100 pL to about 10 nL, about 500 pL to about 600 pL, about 500 pL to about 700 pL, about 500 pL to about 800 pL, about 500 pL to about 900 pL, about 500 pL to about 1 nL, about 500 pL to about 2 nL, about 500 pL to about 3 nL, about 500 pL to about 4 nL, about 500 pL to about 5 nL, about 500 pL to about 6 nL, about 500 pL to about 7 nL, about 500 pL to about 8 nL, about 500 pL to about 9 nL, about 500 pL to about 10 nL, about 1 nL to about 2 nL, about 1 nL to about 3 nL, about 1 nL to about 4 nL, about 1 nL to about 5 nL, about 1 nL to about 6 nL, about 1 nL to about 7 nL, about 1 nL to about 8 nL, about 1 nL to about 9 nL, or about 1 nL to about 10 nL.

For embodiments employing an array of droplets, the number of droplets in the array will depend on the composition and end use of the array. Any suitable number of droplets can be used. Arrays containing from about 2 to many billions of droplets can be made by utilizing a variety of techniques and materials. Increasing the number of droplets in the array can be used to increase the dynamic range of an assay or to allow multiple samples or multiple types of analyte molecules to be assayed in parallel. Generally, the array will comprise between one thousand and one billion droplets per sample to be analyzed. In some cases, the array will comprise greater than one million droplets, greater than ten million droplets, greater than one hundred million droplets, or greater than one billion droplets. For example, in some embodiments, the sample will comprise between 1,000 and 10⁹ droplets. For example, in some embodiments, the array will comprise between about 1,000 and about 50,000, between about 1,000 and about 1,000,000, between about 1,000 and about 10,000, between about 10,000 and about 100,000, between about 100,000 and about 1,000,000, between about 1,000 and about 100,000, between about 50,000 and about 100,000, between about 20,000 and about 80,000, between about 30,000 and about 70,000, between about 40,000 and about 60,000, or about 50,000 droplets.

The methods of the present invention provide ultra-sensitive detection and quantification of target analytes by interrogating a large percentage of droplets with minimal sample loss. In some embodiments, at least 30% of the droplets are detected. In some embodiments, at least 40% of the droplets are detected. In some embodiments, at least 50% of the droplets are detected. In some embodiments, at least 60% of the droplets are detected. In some embodiments, at least 70% of the droplets are detected. In some embodiments, at least 80% of the droplets are detected. In some embodiments, at least 90% of the droplets are detected. In some embodiments, at least 91% of the droplets are detected. In some embodiments, at least 92% of the droplets are detected. In some embodiments, at least 93% of the droplets are detected. In some embodiments, at least 94% of the droplets are detected. In some embodiments, at least 95% of the droplets are detected. In some embodiments, at least 96% of the droplets are detected. In some embodiments, at least 97% of the droplets are detected. In some embodiments, at least 98% of the droplets are detected. In some embodiments, at least 99% of the droplets are detected. In some embodiments, 100% of the droplets are detected.

In some embodiments, at least 30% of the detection probes are detected. In some embodiments, at least 40% of the detection probes are detected. In some embodiments, at least 50% of the detection probes are detected. In some embodiments, at least 60% of the detection probes are detected. In some embodiments, at least 70% of the detection probes are detected. In some embodiments, at least 80% of the detection probes are detected. In some embodiments, at least 90% of the detection probes are detected. In some embodiments, at least 91% of the detection probes are detected. In some embodiments, at least 92% of the detection probes are detected. In some embodiments, at least 93% of the detection probes are detected. In some embodiments, at least 94% of the detection probes are detected. In some embodiments, at least 95% of the detection probes are detected. In some embodiments, at least 96% of the detection probes are detected. In some embodiments, at least 97% of the detection probes are detected. In some embodiments, at least 98% of the detection probes are detected. In some embodiments, at least 99% of the detection probes are detected. In some embodiments, 100% of the detection probes are detected.

In some embodiments, at least 30% of the target analytes in a sample are detected. In some embodiments, at least 40% of the target analytes in a sample are detected. In some embodiments, at least 50% of the target analytes in a sample are detected. In some embodiments, at least 60% of the target analytes in a sample are detected. In some embodiments, at least 70% of the target analytes in a sample are detected. In some embodiments, at least 80% of the target analytes in a sample are detected. In some embodiments, at least 90% of the target analytes in a sample are detected. In some embodiments, at least 91% of the target analytes in a sample are detected. In some embodiments, at least 92% of the target analytes in a sample are detected. In some embodiments, at least 93% of the target analytes in a sample are detected. In some embodiments, at least 94% of the target analytes in a sample are detected. In some embodiments, at least 95% of the target analytes in a sample are detected. In some embodiments, at least 96% of the target analytes in a sample are detected. In some embodiments, at least 97% of the target analytes in a sample are detected. In some embodiments, at least 98% of the target analytes in a sample are detected. In some embodiments, at least 99% of the target analytes in a sample are detected. In some embodiments, 100% of the target analytes in a sample are detected.

The array of droplets may be arranged on a substantially planar surface or, alternatively, in a non-planar three-dimensional arrangement.

Droplets may be stabilized using a surfactant. Any suitable surfactant can be used. In some embodiments, the surfactant is a fluorosurfactant, e.g., 008-FluoroSurfactant, a perfluoropolyether (PFPE)-poly(ethylene glycol) (PEG)-PFPE triblock copolymer, a PFPE-linear polyglycerol hydroxyl (LPG(OH))-PFPE triblock copolymer, a PFPE-poly(methyl glycerol) methoxy (LPG(OMe))-PFPE triblock copolymer, or a combination thereof.

Any suitable concentration of the surfactant may be used. In some embodiments, the surfactant may have a concentration of about 0.01%, about 0.02%, about 0.03%, about 0.04%, about 0.05%, about 0.06%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 5.0%, about 6.0%, about 7.0%, about 8.0%, or higher (e.g., weight percentage).

### IV. Detection

Capture probes, detectable moieties, detection probes, and target analytes can be detected and/or quantified, and the detection and/or quantification can be related to the presence and, optionally, the quantity and/or concentration of target analytes in the sample being tested. In some embodiments, a plurality of capture probes, detectable moieties, detection probes, or target analytes may be detected and/or quantified by spatially segregating the plurality of capture probes, detectable moieties, detection probes, or target analytes into a plurality of droplets (e.g., in a droplet array). In some embodiments, a detector may be configured to detect the capture probes, detectable moieties, detection probes, or target analytes in or at a plurality of droplets (e.g., a droplet array). In some embodiments, the capture probes, detectable moieties, detection probes, or target analytes may be able to produce or be made to produce a detectable signal, for example, fluorescence emission, for the detection of the capture probes, detectable moieties, detection probes, or target analytes. In some cases, the capture probes, detectable moieties, detection probes, or target analytes may be detected using scattering techniques, as described herein.

In some embodiments, non-enzymatic detection methods may be employed. Any suitable non-enzymatic detection method may be used. Non-limiting examples include absorbance, calorimetry (e.g., differential scanning calorimetry (DSC)), circular dichroism, diffraction, electron microscopy (e.g., scanning electron microscopy (SEM), x-ray photoelectron microscopy (XPS)), electron paramagnetic resonance (EPR), electrical transduction methods (e.g., conduction and capacitance), evanescent wave detection, electromagnetic radiation resonance methods (e.g., whispering gallery modes), fluorescence technologies (e.g., fluorescence resonance energy transfer (FRET), time-resolved fluorescence (TRF), fluorescence polarization (FP)), light scattering, luminescent oxygen channeling (LOCI), magnetic transduction effects (e.g., magnetoresistive effect), mass spectroscopy (e.g., matrix assisted laser desorption and ionization (MALDI)), nuclear magnetic resonance (NMR), optical interferometry and other methods based on measuring changes in refractive index, piezoelectric transduction (e.g., quartz crystal microbalance (QCM)), Raman scattering, spectroscopy (e.g., infrared, atomic spectroscopies), scanning probe microscopy (e.g., atomic force microscopy (AFM), scanning tunneling microscopy (STM)), and surface plasmon resonance (SPR).

In some embodiments, indirect detection may be employed. The indirect approach can include, for example, exposing a capture probe, a detectable moiety, a detection probe, or a target analyte to a precursor labeling agent, in which the precursor labeling agent is converted into a labeling agent upon exposure to the capture probe, detectable moiety, detection probe, or target analyte. The labeling agent may comprise a molecule or moiety that can be interrogated and/or detected. The presence or absence of a capture probe, a detectable moiety, a detection probe, or a target analyte at a location (e.g., in a droplet, e.g., in a droplet array) may then be determined by determining the presence or absence of a labeling agent at/in the location. For example, a capture probe, a detectable moiety, a detection probe, or a target analyte may include, be bound to, or associated with an enzymatic label, and the precursor labeling agent molecule may be a chromogenic, fluorogenic, or chemiluminescent enzymatic precursor labeling agent molecule which is converted to a chromogenic, fluorogenic, or chemiluminescent product (each an example of a labeling agent) upon exposure to the converting agent. In this instance, the precursor labeling agent may be an enzymatic label, for example, a chromogenic, fluorogenic, or chemiluminescent enzymatic precursor labeling agent, that upon contact with the enzymatic component, is converted into a labeling agent, which is detectable. In some cases, the chromogenic, fluorogenic, or chemiluminescent enzymatic precursor labeling agent is provided in a droplet maker such that it is present in the droplet or plurality of droplets. In some embodiments, an electrochemiluminescent precursor labeling agent is converted to an electrochemiluminescent labeling agent. In some cases, the enzymatic label may comprise beta-galactosidase, horseradish peroxidase, or alkaline phosphatase.

In some embodiments, a plurality of locations (e.g., droplets) may be addressed, and/or a plurality of capture probes, detectable moieties, detection probes, or target analytes may be detected substantially simultaneously. Simultaneous addressing/detection can be accomplished by using various techniques, including optical techniques (e.g., using a charge coupled device (CCD) detector, charge-injection device (CID), or complementary-metal-oxide-semiconductor detector (CMOS) detector). Any suitable detector may be used in the methods described herein.

### V. Target Analytes

As would be appreciated by a person of ordinary skill in the art, a large number of target analytes can be detected and, optionally, quantified using the methods of the invention. Any suitable target analyte can be investigated using the methods of the invention. The target analytes listed below are provided as non-limiting examples. The target analyte may be naturally occurring or synthetic.

For example, in some embodiments, the target analyte is, without limitation, a protein (e.g., an antibody, a cytokine (e.g., an interleukin (e.g., IL-1, IL-2, IL-3, IL-4, IL-5, IL-6, IL-7, IL-7, IL-9, IL-10, IL-11, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, or IL-36), a lymphokine, a monokine, an interferon (IFN, e.g., IFN-beta and IFN-gamma), a colony stimulating factor (e.g., CSF, G-CSF, GM-CSF, and the like), a chemokine, a tumor necrosis factor (TNF, including TNF-alpha and TNF-beta), a bone morphogenetic protein (BMP), and the like), a receptor (e.g., an interleukin receptor, a receptor tyrosine kinase, and the like), a ligand, an enzyme (e.g., a polymerase, a cathepsin, a calpain, an aminotransferase (e.g., aspartate aminotransferase (AST) or alanine aminotransferase (ALT)), a protease (e.g., a caspase), a lipase, an oxidoreductase, a kinase, nucleotide cyclases, a transferase, a hydrolase, a lyase, an isomerase, and the like), or a prion), a nucleic acid (e.g., DNA, RNA (e.g., microRNA), or a modified nucleic acid), a polysaccharide, a lipid, a cell (e.g., a prokaryotic cell (e.g., a bacterium (e.g., E. coli)) or a eukaryotic cell (e.g., a fungal cell or a human cell), including tumor cells), a fatty acid, an extracellular vesicle, a glycoprotein, a glycan, a biomolecule, a therapeutic agent (e.g., an antibody, a fusion protein (e.g., an Fc fusion protein), a cytokine, a soluble receptor, and the like), an organism (e.g., a pathogen), a virus (e.g., a parvovirus (e.g., an adeno-associated virus (AAV)), a retrovirus, a herpesvirus, an adenovirus, a lentivirus, and the like), or a small molecule. The target analyte may be a toxin. In some embodiments, the target analyte may be post-translationally modified (e.g., phosphorylated, methylated, glycosylated, ubiquitinated, and the like).

In some embodiments, the target analyte has a molecular weight of greater than about 5000 Da, greater than about 10,000 Da, greater than about 20 kDa, greater than about 30 kDa, greater than about 40 kDa, greater than about 50 kDa, greater than about 100 kDa, greater than about 200 kDa, or greater than about 300 kDa.

In some embodiments, the target analyte is a small molecule. Any suitable small molecule may be detected and, optionally, quantified using the methods of the invention. For example, in some embodiments, the small molecule is an organic compound, an inorganic compound, a steroid (e.g., an androgen/anabolic steroid (e.g., testosterone, 4-hydroxytestosterone, 11-ketotestosterone, boldenone, clostebol, 4-androstenediol, 4-dehydroepiandrosterone (4-DHEA), 5-androstendione, 5-dehydroandrosterone (5-DHA), adrenosterone, adrostenediol, atamestane, cloxotestosterone, quinbolone, silandrone, stanolone, 1-testosterone, nandrolone, or derivatives thereof), an estrogen (e.g., estradiol, 2-hydroxyestradiol, 4-hydroxyestradiol, 4-methoxyestradiol, estrazinol, estrofurate, ethinylestradiol, mestranol, methylestradiol, moxestrol, quinestol, estrone, estriol, or derivatives thereof), a progestogen (e.g., progesterone, quingestrone, retroprogesterone, dydrogesterone, trengestone, hydroxyprogesterone, or derivatives thereof), a corticosteroid (e.g., a glucocorticoid or a mineralcorticoid, including, e.g., cortisol, cortisone, prednisone, prednisolone, methylprednisolone, dexamethasone, betamethasone, triamcinolone, fludrocortisone acetate, deoxycorticosterone acetate, or derivatives thereof), a neurosteroid (e.g., a cholestane (e.g., 25-hydroxycholesterol), an androstane (e.g., 3α-androstanediol, etiocholanediol, and the like), a pregnane (e.g., 3α-DHP, allopregnanedione, or pregnanedione), a steroid ester, and the like), a hormone (e.g., melatonin, thyroxine, TRH, vasopressin, eicosanoids (e.g., arachidonic acid, lipoxins, thromboxanes, leukotrienes, and prostaglandins (e.g., prostaglandin E2)), steroids as described above, and plant hormones (e.g., abscisic acid, auxin, cytokinin, ethylene, and gibberellin)), a hapten, a biogenic amine (e.g., a monoamine neurotransmitter (e.g., histamine, serotonin, norepinephrine, epinephrine, and dopamine), a trace amine, a thyronamine, tryptamine, trimethylamine, agmatine, adaverine, putrescine, spermine, spermidine, and the like), an antibiotic (e.g., vancomycin, lincosamides (e.g., clindamycin and lincomycin), quinolones (e.g., ciprofloxacin and the like), sulfonamides (e.g., mafenide and the like), macrolides (e.g., azithromycin and clarithromycin), lipopeptide (e.g., daptomycin), dalbavacin, fusidic acid, oxazolidinones (e.g., linezolid), tetracyclines (e.g., minocycline, tetracycline, doxycycline, and the like), mupirocin, oritavancin, tedizolid, telavancin, tigecycline, aminoglycosides (e.g., amikacin, gentamycin, neomycin, kanamycin, tobramycin, and streptomycin), monobactams, carbapenems (e.g., ertapenem, doripenem, imipenem, and meropenem), ceftazidime, tazobactam, penicillins (e.g., penicillin, temocillin, and the like), rifaximin, and cephalosporins (e.g., cefixime, ceftobiprole, and ceftaroline)), a mycotoxin (e.g., aflatoxins, ochratoxins, citrinins, patulins, and fusarium toxins), a cyanotoxin (e.g., microcystin, nodularin, cylindrospermopsin, saxitoxin, neosaxitoxin, and gonyautoxin), an organic pollutant, a nucleotide, an amino acid, a peptide, a monosaccharide (e.g., glucose, fructose, or galactose), a drug residue (e.g., chloramphenicol, clenbuterol, and tylosin), a pesticide residue (e.g., cypermethrin, triazophos, methyl-parathion, fenpropathrin, carbofuran, thiacloprid, chlorothalonil, and carbendazim), or a secondary metabolite (e.g., an alkaloid, a terpenoid, a steroid, a flavonoid, a glycoside, a natural phenol (e.g., resveratrol), a phenazine, a biphenyl, a dibenzofuran, a polyketide, a fatty acid synthase product, a nonribosomal peptide (e.g., vancomycin, ramoplanin, and the like), or a polyphenol).

In some embodiments, the small molecule has a molecular weight of less than about 5000 Da, less than about 4500 Da, less than about 4000 Da, less than about 3500 Da, less than about 3000 Da, less than about 2500 Da, less than about 2000 Da, less than about 1500 Da, less than about 1000 Da, less than about 900 Da, less than about 800 Da, less than about 700 Da, less than about 600 Da, less than about 500 Da, less than about 400 Da, less than about 300 Da, less than about 200 Da, or less than about 100 Da.

In some embodiments, the small molecule is an organic molecule, including small organic compounds having a molecular weight of more than 100 and less than about 2,500 Da. In some embodiments, the small organic compound may include any suitable functional group, including an amine, carbonyl, hydroxyl, or carboxyl group, optionally at least two of the functional chemical groups. A small molecule may include cyclical carbon or heterocyclic structures and/or aromatic or polyaromatic structures substituted with one or more of the above functional groups.

For example, in some embodiments, the methods may include detecting and, optionally, quantifying, about 1, about 2, about 3, about 4, about 5, about 6, about 7, about 8, about 9, about 10, about 11, about 12, about 14, about 16, about 18, about 20, or more different target analytes.

In some embodiments, the target analyte may be a nucleic acid. A nucleic acid may be captured or detected with a complementary nucleic acid fragment (e.g., an oligonucleotide). For example, a detection probe for a nucleic acid target analyte may be or include a complementary oligonucleotide. A detectable moiety (e.g., an enzyme) may bind to a different portion of the nucleic acid target analyte, e.g., using an oligonucleotide that is complementary to a different portion of the nucleic acid target analyte.

### VI. Samples

Any suitable sample may be used in the context of the present invention. For example, in some embodiments, the sample is a liquid sample (e.g., a biological sample or an environmental sample). Exemplary biological samples include, without limitation, body fluids, body tissue (e.g., tumor tissue), cells, or other sources. Exemplary body fluids include, without limitation, e.g., lymph, whole blood (including fresh or frozen), plasma (including fresh or frozen), serum (including fresh or frozen), a blood fraction containing peripheral blood mononuclear cells, urine, saliva, semen, sweat, lacrimal fluid, synovial fluid, cerebrospinal fluid, feces, mucous, vaginal fluid, and spinal fluid. Samples also include breast tissue, renal tissue, colonic tissue, brain tissue, muscle tissue, synovial tissue, skin, hair follicle, bone marrow, tumor tissue, a tissue lysate or homogenate, and an organ lysate or homogenate. Methods for obtaining tissue biopsies and body fluids from mammals are well known in the art. In other embodiments, the sample may be an environmental sample, e.g., a water sample, soil sample, air sample, extraterrestrial materials, or the like.

The volume of the fluid sample analyzed may potentially be any amount within a wide range of volumes, depending on a number of factors such as the number of capture probes used/available, the number of detection probes, and the like. As non-limiting examples, the sample volume may be about 0.01 μl, about 0.1 μl, about 1 μl, about 5 μl, about 10 μl, about 100 μl, about 1 ml, about 5 ml, about 10 ml, or more. In some cases, the volume of the fluid sample is between about 0.01 μl and about 10 ml, between about 0.01 μl and about 1 ml, between about 0.01 μl and about 100 μl, or between about 0.1 μl and about 10 μl.

In some embodiments, a fluid sample may be diluted prior to use in a method described herein. For example, in embodiments where the source of a target analyte is a body fluid (e.g., blood, plasma, or serum), the fluid may be diluted with an appropriate diluent (e.g., a buffer such as PBS buffer). A fluid sample may be diluted about 2-fold, about 3-fold, about 4-fold, about 5-fold, about 6-fold, about 10-fold, about 50-fold, about 100-fold, or greater, prior to use. The sample may be added to a liquid comprising the plurality of capture probes or detectable moieties, or the plurality of capture probes or detectable moieties may be added to the sample directly or in a liquid.

### VII. Detection Probes

Any suitable detection probe may be used in the context of the present invention. For example, in some embodiments, the detection probe is an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a molecularly-imprinted polymer, a receptor, a polypeptide, a nucleic acid, or a small molecule.

In some embodiments, the detection probe is covalently or non-covalently linked to a detectable moiety or to a member of a non-covalent affinity binding pair.

### VIII. Capture Probes and Capture Ligands

Any suitable capture probes can be used in the context of the invention, including, without limitation, beads (e.g., paramagnetic beads, silica beads, or hydrogel beads), nanotubes, polymers, or the like. Suitable beads include, but are not limited to, paramagnetic beads, plastic beads, ceramic beads, glass beads, silica beads, hydrogel beads, polystyrene beads, methylstyrene beads, acrylic polymer beads, carbon graphited beads, titanium dioxide beads, latex or cross-linked dextrans such as SEPHAROSE beads, cellulose beads, nylon beads, cross-linked micelles, and TEFLON® beads. In particular embodiments, the bead is a paramagnetic bead. The beads may be substantially spherical or non-spherical.

In some embodiments, capture ligands or immobilized target analytes may either be directly synthesized on the capture probes (e.g., beads), or they may be made and then attached after synthesis. In some embodiments, linkers are used to attach the capture ligands or immobilized target analytes to the capture probes (e.g., beads), for example, to allow both good attachment, sufficient flexibility to allow good interaction with the target molecule, and to avoid undesirable binding reactions.

As is known in the art, many classes of chemical compounds are currently synthesized on solid supports, such as peptides, organic moieties, and nucleic acids. It is a relatively straightforward matter to adjust the current synthetic techniques to capture probes (e.g., beads).

In some embodiments, capture ligands or immobilized target analytes are obtained or synthesized first, and then covalently attached to the capture probes (e.g., beads). As will be appreciated by those in the art, this will be done depending on the composition of the capture ligands and the capture probes (e.g., beads). The functionalization of solid support surfaces such as certain polymers with chemically reactive groups such as thiols, amines, carboxyls, and the like is generally known in the art. Accordingly, “blank” capture probes (e.g., beads) may be used that have surface chemistries that facilitate the attachment of the desired functionality. In certain embodiments, capture ligands or immobilized target analytes can be covalently attached to capture probes (e.g., beads) using any suitable chemical reaction, e.g., cycloaddition (e.g., an azide-alkyne Huisgen cycloaddition (e.g., a copper(I)-catalyzed azide-alkyne cycloaddition (CuAAC) or a strain-promoted azide-alkyne cycloaddition (SPAAC))), amide or thioamide bond formation, a pericyclic reaction, a Diels-Alder reaction, sulfonamide bond formation, alcohol or phenol alkylation, a condensation reaction, disulfide bond formation, or a nucleophilic substitution.

In some instances, a capture probe, an immobilized target analyte, a detection probe, or a capture ligand may include a conjugating moiety. A conjugating moiety includes at least one functional group that is capable of undergoing a conjugation reaction, for example, any conjugation reaction described in the preceding paragraph. The conjugation moiety can include, without limitation, a 1,3-diene, an alkene, an alkylamino, an alkyl halide, an alkyl pseudohalide, an alkyne, an amino, an anilido, an aryl, an azide, an aziridine, a carboxyl, a carbonyl, an episulfide, an epoxide, a heterocycle, an organic alcohol, an isocyanate group, a maleimide, a succinimidyl ester, a sulfosuccinimidyl ester, a thiol, or a thioisocyanate group.

In some embodiments, a capture probe may be detectably labeled. For example, in multiplexed assays as described herein, a first population of capture probes may be detectably labeled with a first label, and a second population of capture probes may be detectably labeled with a second label, such that the first population and the second population are distinguishable (also referred to herein as “distinguishably labeled”). Any suitable label can be used. For example, the label may be a reporter dye (e.g., a fluorescent dye, a chromophore, or a phospho), or a mixture thereof). By varying both the composition of the mixture (i.e., the ratio of one dye to another) and the concentration of the dye (leading to differences in signal intensity), matrices of unique tags may be generated. Capture probes (e.g., beads) can be labeled using any suitable approach, for example, by covalently attaching the label (e.g., a dye) to the surface of the capture probes, or alternatively, by entrapping the label (e.g., a dye) within the capture probe. Such dyes may be, for example, covalently attached to the surface of a capture probe (e.g., a bead), for example, using any of the conjugation approaches described above or herein. Suitable dyes for use in the invention include, but are not limited to, ALEXA FLUOR® dyes, CY® dyes, DYLIGHT® dyes, fluorescein, rhodamine, tetramethylrhodamine, eosin, erythrosin, coumarin, methyl-coumarins, pyrene, Malachite green, fluorescent lanthanide complexes, including those of europium and terbium, stilbene, Lucifer Yellow, CASCADE BLUE™, TEXAS RED®, and others known in the art (e.g., as described in The Molecular Probes Handbook, 11^{th} Ed., 2010).

In some embodiments, the methods described herein may involve use of a capture ligand. Any suitable capture ligand can be used in the context of the invention. Exemplary capture ligands include an antibody (e.g., a full-length antibody (e.g., an IgG, IgA, IgD, IgE, or IgM antibody) or an antigen-binding antibody fragment (e.g., an scFv, an Fv, a dAb, a Fab, an Fab′, an Fab′₂, an F(ab′)₂, an Fd, an Fv, or an Feb)), an aptamer, an antibody mimetic (e.g., an affibody, an affilin, an affimer, an affitin, an alphabody, an anticalin, an avimer, a DARPin, a fynomer, a Kunitz domain peptide, a monobody, or a nanoCLAMP), a polypeptide, an antibody IgG binding protein (e.g., protein A, protein G, protein L, and recombinant protein A/G), a nucleic acid, or a small molecule. For example, in some embodiments, a capture ligand binds to an Fc region of an antibody. A capture ligand can be covalently or non-covalently attached to a capture probe (e.g., a bead) using any approach known in the art or described herein.

### IX. Detectable Moieties

Any suitable detectable moiety may be used in the context of the invention. For example, a variety of enzymatic labels or colored labels (for example, metallic nanoparticles (e.g., gold nanoparticles), semiconductor nanoparticles, semiconductor nanocrystals (e.g., quantum dots), spectroscopic labels (for example, fluorescent labels), and radioactive labels) may be used in the methods described herein.

Depending upon the particular assay format, the detectable moiety can be indirectly attached, for example, to a target analyte or to a detection probe. In some embodiments, the amount of the detection moiety in a step of a method is proportional to the amount of the target analyte in the sample. The presence of the detectable moiety can be detected using suitable detection systems, for example, optical detectors (for example, intensified CCD cameras), or any other suitable detectors known in the art.

In one embodiment, the detectable moiety is an enzymatic label. In such embodiments, a chromogenic, fluorogenic, or chemiluminescent enzyme substrate may be contacted with the enzyme to produce a detectable product. Suitable chromogenic, fluorogenic, or chemiluminescent enzyme substrates are known. Thus, any known chromogenic, fluorogenic, or chemiluminescent enzyme substrate capable of producing a detectable product in a reaction with a particular enzyme can be used in the present invention.

For example, in some embodiments in which the analyte is detected or quantified using a method as described herein in which the enzyme label is β-galactosidase, the enzyme substrate added to the array can be a β-galactosidase substrate such as resoruffn-β-D-galactopyranoside (RGP) or fluorescein di(β-d-galactopyranoside).

### X. Kits and Articles of Manufacture

The invention provides kits and articles of manufacture for measuring a concentration of a target analyte (e.g., a small molecule) in a fluid sample. The article or kit may include, for example, a plurality of capture probes (e.g., beads, e.g., paramagnetic beads), detection probes, capture ligands, detectable moieties, and/or a device for producing droplets (e.g., a microfluidic device as described herein). The plurality of capture probes (e.g., beads) may have an average diameter between about 0.1 micrometer and about 100 micrometers, and the device for producing the droplets may be may be configured such that only zero or one beads is contained in a droplet.

The kits and articles of manufacture described herein may be configured for carrying out any of the methods or assays as described herein, e.g., in the Examples. The kits and articles of manufacture may include any of the droplets (e.g., droplet arrays) described herein.

The plurality of capture probes (e.g., beads) provided may have a variety of properties and parameters, as described herein. For example, the beads may be magnetic.

In some embodiments, the kit or article may include a detectable moiety, as described herein. The kit may further include an enzyme substrate.

The kit or article may include buffers, diluents, solvents, or other reagents for carrying out the methods described herein.

In some embodiments, the kit or article may include instructions for use of components described herein. That is, the kit or article can include a description of use of the capture probes (e.g., beads) and droplets, for example, for use with a system to determine a measure of the concentration of target analyte(s) in a fluid sample. As used herein, “instructions” can define a component of instruction and/or promotion, and typically involve written instructions on or associated with packaging of the invention. Instructions also can include any oral or electronic instructions provided in any manner such that a user of the kit or article will clearly recognize that the instructions are to be associated with the kit or article. Additionally, the kit or article may include other components depending on the specific application, as described herein.

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Materials and Methods

The following materials and methods were used in the Examples below.

### Microfluidics Device Fabrication

All microfluidics devices used in this paper are fabricated using polydimethylsiloxane (PDMS) with the standard soft lithographic methods. Channels then undergo a hydrophobic surface treatment by flowing through Aquapel (PPG, Pittsburgh, Pa.) followed by pressured nitrogen blow.

### Droplet Generation

Magnetic bead complexes were concentrated using a strong magnet and the supernatant was removed completely using a 20 μl pipette. Beads were resuspended in 2 μl solution containing 1.7 μl of reagent and 0.3 μl (15% v/v) density gradient solution Optiprep (Sigma) in a PCR tube. The solution was then mixed for >30 times using a 2 μl pipette.

In the meantime, a 1 ml syringe (BD Luer-Lok 1-ml Syringe, Beckton Dickinson) containing 300 μl of HFE 7500 was prepared for sample loading with needle ( ) and PE/2 tubing (Scientific Commodities, Inc.) attached. HFE 7500 was pushed manually until the fluid was approximately 0.5 cm away from the tip of the tubing. Immerse the tubing's tip into the magnetic bead solution and begin withdrawal by slowly pulling the syringe's plunger. This process was repeated by immersing the tip into 1 μl HFE 7500 oil. The syringe was then loaded onto the syringe pump and ready for injection. The other syringe was prepared by adding HFE 7500 with 2% Fluoro-surfactant (008-FluoroSurfactant, Ran Biotechnologies).

Tubings were inserted into the corresponding inlets on the device. VWR gel loading pipet tip was inserted into the outlet for droplet collection. The pump was then started with flow rate of 120 ul/hr for the oil phase and 80 ul/hr for the sample. Mono-dispersed droplets with a diameter of 14um were generated at rates ˜20,000 HZ under 1 min for a total of ˜1.4 million droplets. Droplets were then transferred from gel loading pipet tip to PCR tube for incubation.

### Droplet Reinjection for Imaging

A 1 ml syringe containing 300 μl of HFE7500 with 2% surfactant was prepared. Due to small droplets volume, droplets were loaded into the syringe the same way as the sample loading process described above. Oil with 2% surfactant is pushed manually until it was approximately 0.5 cm away from the tip of the tubing. Immerse the tubing's tip into the bottom of the droplet solution and begin withdrawal by slowly pulling the plunger until all droplets were inside the tubing. Then the syringe was loaded onto the syringe pump. The tubing was taped to the side of the syringe pump while the tip of the tubing points downward. Due to gravity and density difference between droplets and oil, droplets will flow upwards until a clear separation appears after ˜3 minutes so that oil is near the tip of the tubing. This technique allows us to closely pack the droplets, which is desirable for droplet loading.

In the meantime, the imaging chamber was flushed with oil containing 2% surfactant prior to droplet loading. Droplet tubing was inserted into the inlet of the microfluidics device and began pumping with a flow rate of 100 μl/hr until all droplets are loaded. Unplug the tubing from the microfluidics device and begin imaging.

### Image Analysis

Using the brightfield image, droplet regions were isolated using Laplacian of Gaussian edge detection followed by morphological region filling. A watershed algorithm was applied to large regions to separate any partially connected regions. Using the fluorescent image of the beads the beads were located and matched to a droplet using a nearest neighbor algorithm, followed by confirming overlap. Using the location of each droplet the median intensity value of that location within the enzyme fluorescence image was taken. All droplets were matched with 5 nearest neighbors that did not contain a bead. If a droplet had too few neighbors within one diameter, it was excluded from analysis. Once all droplets were matched with neighbors the median intensity value of the neighbors could be subtracted from the intensity value to give a relative-to-background intensity value for each droplet.

The distribution of the droplets not containing beads closely resembles the distribution of droplets with beads but without enzyme activity allowing it to be used to approximate the distribution of “off” droplets. The intensity of the droplets not containing beads closely followed a Gaussian distribution allowing for a fit to be applied to determine the mean and standard deviation. Using the fitted Gaussian, a cutoff was set at 10 standard deviations above the mean, any bead-containing droplet with an intensity value above this cutoff was determined to be “on”. Fraction on was then calculated to be the number of “on” beads over the total number of beads, using this the average number of enzymes per bead (AEB) was determined via Poisson statistics.

### Example 2: Single Molecule Detection of Proteins using Droplet Arrays

Described herein are single molecule assays using droplet microfluidics. These assays enable high sensitivity measurements with improved multiplexing capabilities compared to existing methods and are also more amenable to miniaturization.

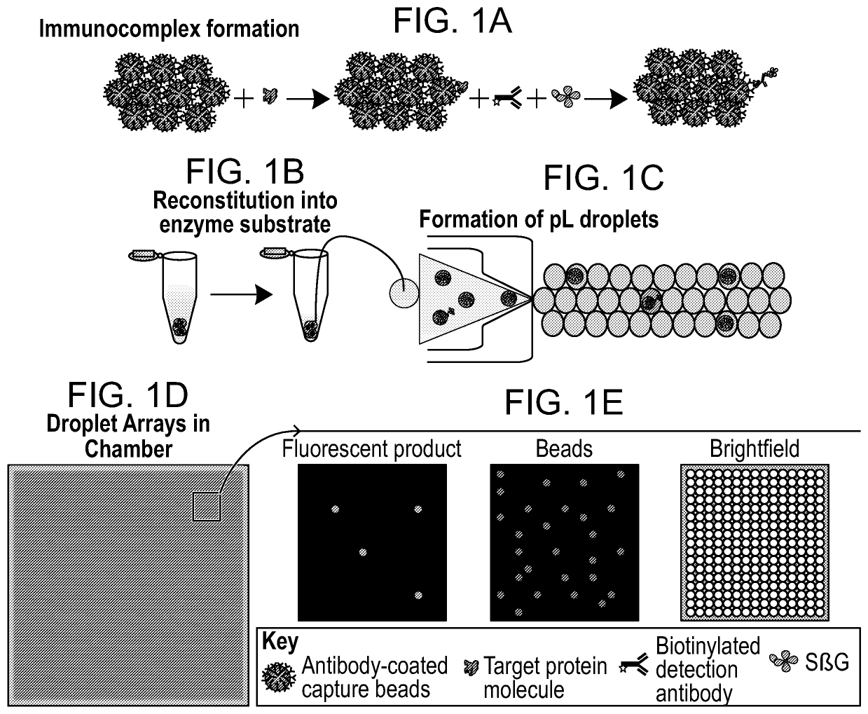

Single molecule detection of proteins using droplet arrays is a bead-based immunoassay method in which the beads are isolated in pL-sized droplets and loaded onto a chamber, forming droplet arrays, for analysis (FIGS. 1A-1E). More specifically, antibody-coated paramagnetic beads are added to a sample containing the target molecule (FIG. 1A). The target molecule is then labeled with a biotinylated detection antibody and streptavidin-β-galactosidase (SBG), forming an enzyme-labeled immunocomplex. The beads are then re-suspended in a small volume (2 μl) of substrate, for example, fluorescein di-β-D-galactopyranoside (FDG) (FIG. 1B) and the mixture is partitioned into pL droplets such that most droplets contain either zero beads or one bead (FIG. 1C). The droplets are then loaded onto a chamber in a monolayer to form droplet arrays (FIG. 1D). Images in three channels are obtained to identify i) the droplets, ii) the beads, and iii) the fluorescent product and thus the “on” droplets (FIG. 1E). The signal output is measured using the unit of average enzymes per bead (AEB).

As illustrated in FIG. 1A, an excess number of antibody-coated capture beads are added to a sample containing low concentrations of target analytes. Poisson statistics dictates that either one or zero target analyte molecule will bind to the beads. For example, as depicted in Table 1, a 100 μL blood sample containing 0.1 fM of the target protein has about 6,000 protein molecules. If about 500,000 antibody-coated beads are incubated with the blood sample, the majority of beads will bind zero protein molecules while a small number of beads will bind one protein molecule. A negligible number of beads will bind more than one protein molecule based on the Poisson distribution. Thus, the number of beads that are used to capture single protein molecules, and the number of beads that are subsequently analyzed, will have a strong impact the sensitivity of the assay (Table 1). As shown below in Table 1, reducing the number of beads results in an increase in the average number of molecules per bead, and thus an increase in the number of positive events observed upon analyzing a given number of beads.

TABLE 1
Effect of bead number on analysis of an 0.1
fM solution, 100 μl, with 6,000 molecules

| | | Analyze: | Analyze | Analyze |
|---|---|---|---|---|
| No. of | Molecules/ | 20,000 | 100,000 | all beads |
| Beads | bead | beads (+events) | beads (+events) | (+events) |
| 500,000 | 0.012 | 240 | 1,200 | 6,000 |
| 200,000 | 0.03 | 600 | 3,000 | 6,000 |

The methods of the current disclosure provide three major advantages compared to conventional single-molecule detection methods. The first is reduction in the number of beads that are used. Using less beads for the assay, the ratio of “on” droplets to the total number of beads is increased. A lower number of beads, will result in a higher “fraction on” (fₒₙ, the number of positive events over the total number of beads), thereby leading to a higher signal. The second is the digital readout system, in which the beads are trapped in pL droplets instead of femtoliter-sized wells. The third is the substrate (FDG) with increased stability in droplets. Other enzymes and substrates may also be used for the method disclosed herein, including horseradish peroxidase (HRP) and alkaline phosphatase. By implementing these changes, the current droplet-based methods increase the sensitivity of single-molecule detections.

### Example 3: Theoretical Calculations of Detection Sensitivity

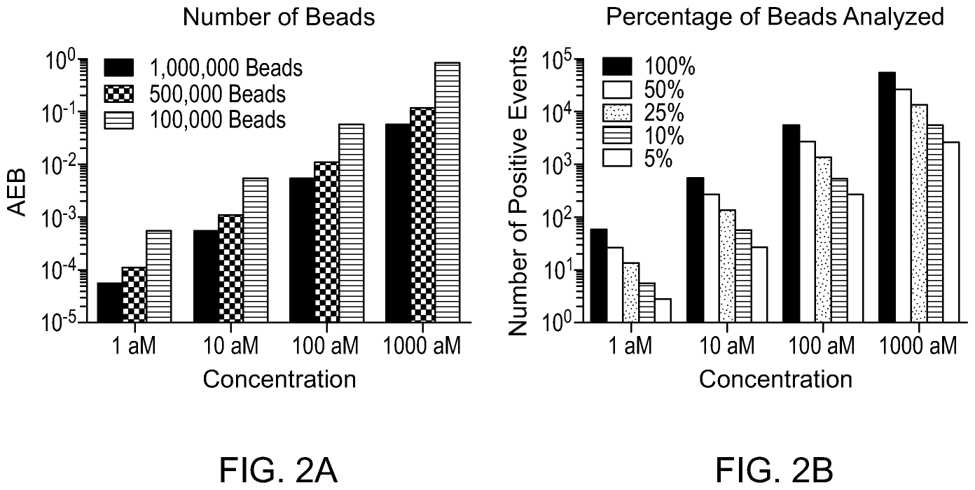

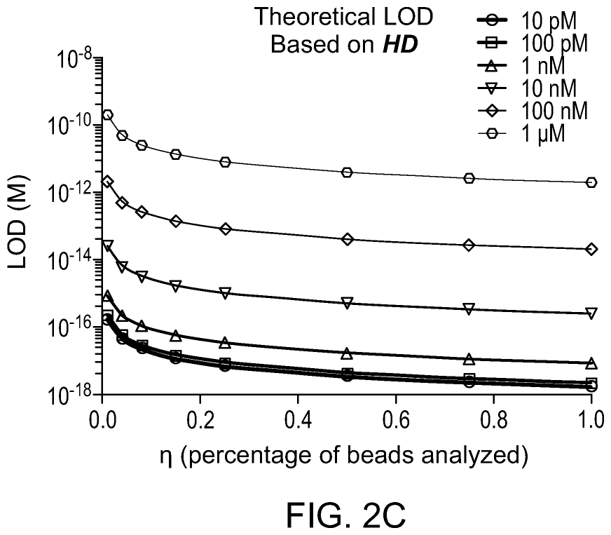

Two important parameters for digital ELISA assays are the number of beads that are used and the percentage of beads that are analyzed. These two parameters can be optimized to achieve maximal sensitivity. The first parameter, the number of beads used, is important since it will determine the fₒₙ and the AEB (average enzymes per bead). In 100 μl of a 10 aM sample there are approximately 600 molecules. Thus, when 1,000,000, 500,000, and 100,000 beads are used, the theoretical AEB is 0.0006, 0.0012, and 0.0060, respectively. As a result, using fewer beads will lead to a higher fₒₙ and AEB (FIG. 2A). Analyzing all of the beads may result in more complex systems and instrumentation that are not amenable for routine or rapid use. Therefore, reducing the number of beads used achieves improved sensitivity since the measured signal will be higher.

The second parameter, the percentage of beads that are analyzed, is important since at ultra-low numbers of molecules, it is essential to measure as many positive events as possible to reduce the measurement uncertainty (FIG. 2B). For example, in 100 μl of a 10 aM sample there are approximately 600 molecules. If 100% of the beads are analyzed, the digital measurement is 600 positive events. If 10% of the beads are analyzed, the digital measurement is 60 positive events. Thus, increasing the percentage of beads analyzed should enable more sensitive detection since the uncertainty in the measurement is reduced.

To understand how the percentage of beads that are analyzed affects the detection limit of Simoa immunoassays, theoretical calculations were performed. The process can be described by three sequential binding steps: (1) capture antibody (cAb) binds target protein analyte (S), forming complex 1, (2) complex 1 binds detection antibody (dAb), forming complex 2, and (3) complex 2 binds streptavidin beta-galactosidase (SBG), forming complex 3. Finally, the amount of complex 3 is measured using a digital readout system.

$$\begin{matrix}Step 1 \text{:} & \\ cAb + S {\to }^{kd 1} complex 1 & ( 1 ) \\ Step 2 \text{:} & \\ complex \#1 + dAb {\to }^{kd 2} complex 2 & ( 2 ) \\ Step 3 \text{:} & \\ complex \#2 + SBG {\to }^{kd 3} complex 3 & ( 3 )\end{matrix}$$

The concentrations of complexes 1-3 can be calculated based on the equilibrium of each reaction step, assuming maximum reaction efficiency is reached.

$$\begin{matrix}capture + ligand {\to }^{KD} complex & ( 4 ) \\ {[ cap t u r e ]}_{total} = [ capture ] + [ complex ] & ( 5 ) \\ {[ ligand ]}_{total} = [ ligand ] + [ complex ] & ( 6 ) \\ KD = \frac{[ capture ] [ ligand ]}{[ complex ]} & ( 7 )\end{matrix}$$

Substituting equations (5) and (6) into equation (7):

$$K D = \frac{\begin{matrix}\{ {[ cap t u r e ]}_{total} - [ complex ] \} \\ \{ {[ ligand ]}_{total} - [ complex ] \}\end{matrix}}{[ complex ]} \qquad ( 8 )$$

Calculating [Complex]:

$$[ complex ] = 0.5 \{ KD + {[ cap t u r e ]}_{total} + {[ ligand ]}_{total} - \sqrt{\begin{matrix}{( KD + {[ cap t u r e ]}_{total} + {[ ligand ]}_{total} )}^{2} - \\ {{4 [ cap t u r e ]}_{total} [ ligand ]}_{total}\end{matrix}} \} \qquad ( 9 )$$

For each step (1-3), the concentration of complexes 1-3 can be determined using equation (9) by substituting the values of [capture]ₜₒₜₐₗ, [ligand]ₜₒₜₐₗ, and KD:

For step 1:

[capture]ₜₒₜₐₗ=[cAb]ₜₒₜₐₗ=[beads]*250,000

[ligand]ₜₒₜₐₗ=[S]ₜₒₜₐₗ

KD=kd1

For step 2:

[capture]ₜₒₜₐₗ=[complex 1]

[ligand]ₜₒₜₐₗ=[dAb]ₜₒₜₐₗ

KD=kd2

For step 3:

[capture]ₜₒₜₐₗ=[complex 2]

[ligand]ₜₒₜₐₗ=[SBG]ₜₒₜₐₗ

KD=kd3

[complex 3] can be solved with [S]ₜₒₜₐₗ as variable. Since the assay is Poisson noise limited, the boundary condition for the LOD is:

Poisson noise limited LOD={[complex #3]*[volume]*η}^{-0.5}≥10% (14)

Where η is the bead loading efficiency and the Poisson noise limited LOD is defined as [S]ₜₒₜₐₗ that meets above boundary condition. FIG. 2C shows the Poisson noise limited LOD and η at different KD values. A higher η leads to a lower LOD, and thus an improvement in sensitivity. The sensitivity of the assay can be improved by about 10 fold, regardless of the KD value, when η from 5% to 50%. Furthermore, a lower KD value leads to improved sensitivity.

Additional parameters of the assay can be controlled to facilitate single molecule detection. For example, to ensure that the fluorescent product of the enzyme-substrate reaction is detectable, the enzyme turnover rate, the substrate concentration, the size of the droplet, and the reaction time can be varied. As illustrated in Table 2, the fluorescent product concentration will be in the nanomolar (nM) range, which is high enough to be easily detectable using a charge-coupled device (CCD) camera.

TABLE 2
Exemplary enzyme-substrate parameters

| Enzyme turnover rate | 600 molecules/second |
|---|---|
| Substrate (e.g., resorufin | 100 μM |
| β-D-galactopyranoside (RGP)) concentration | |
| Droplet size | 30 μm diameter - 14 pL; |
| | 15 μm diameter - 1.8 pL |
| Substrate concentration in droplets after 2 | 30 μm diameter - 8.5 nM; |
| min | 15 μm diameter - 66 nM |

### Example 4:. Detection of Target Analyte Using Droplet Array Assays

### A. Device Design and Fabrication

Antibody-coated capture beads were added in excess to a sample containing low concentrations of target analyte molecules. Poisson statistics dictate that either one or zero target protein molecules will bind to each bead. The beads were then washed and incubated with a biotinylated detection antibody. The beads are then washed and incubated with streptavidin-β-galactosidase (SβG), forming an enzyme-labeled immunocomplex. In some assay formats, the beads, sample and detection antibody are added simultaneously and then washed, followed by addition of SβG. In other assay formats, beads, sample, detection antibody, and SβG are added simultaneously and then washed. The beads were then loaded into droplets in the presence of fluorogenic substrate.

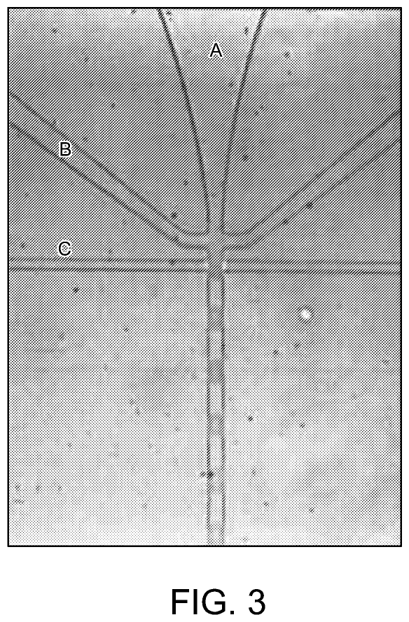

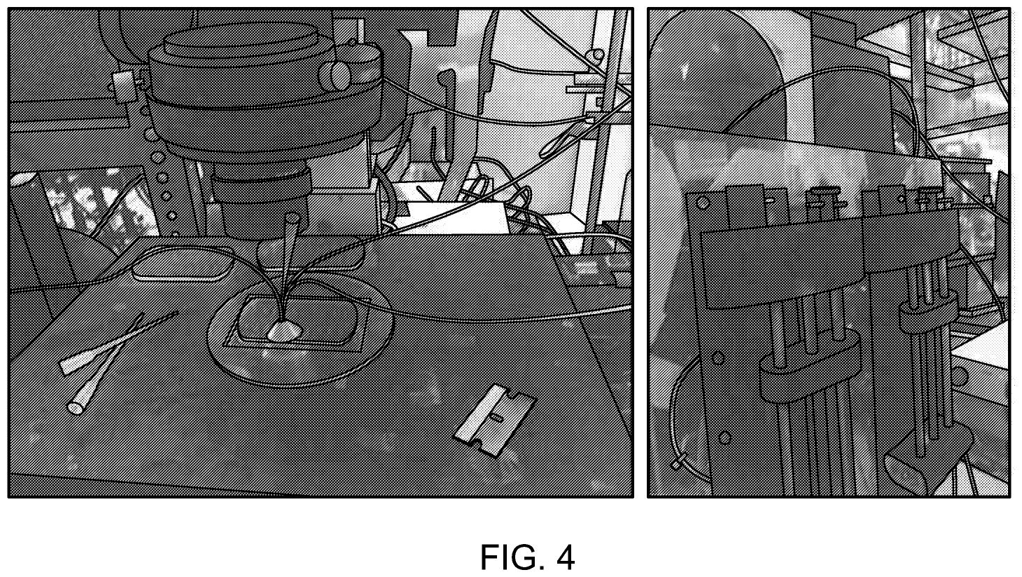

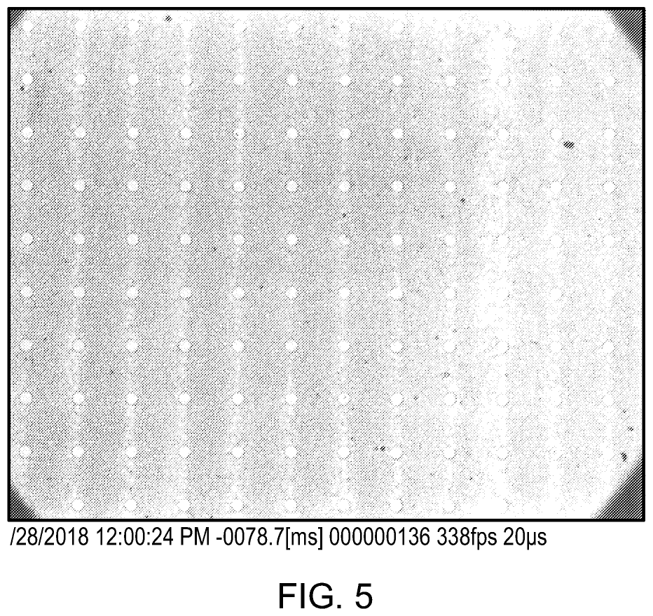

To generate droplets, a flow-focusing device was used, which allows generation of droplets with desired size at the rate of thousands of droplets per second. Droplets were stabilized by 2% (wt/wt) surfactant (Ran Biotechnologies, item number: 008-FluoroSurfactant) in HFE 7500 (3M™ NOVEC™ 7500 engineered fluid) oil. In other examples, the surfactant can include perfluoropolyether (PFPE)-poly(ethylene glycol) (PEG)-PFPE triblock copolymers, PFPE-linear polyglycerol hydroxyl (LPG(OH))-PFPE, and/or PFPE-poly(methyl glycerol) methoxy (LPG(OMe))-PFPE. A density gradient medium (such as OPTIPREP™ iodixanol solution) can also added to the beads, and was used in this Example. The purpose of the density gradient medium is to evenly distribute the beads in solution and reduce or prevent bead aggregation, facilitating isolation of a single bead in a droplet. The beads were co-flowed with the enzyme substrate such that one bead was encapsulated inside each individual droplet along with the desired volume of enzyme substrate. This was achieved by adjusting the flow rate. Three different inlets were used to control the flow rate (FIGS. 3 and 4). The first inlet was for the beads, some of which contain an enzyme-labeled immunocomplex. The second inlet was for the enzyme substrate. The third inlet was for the oil. The dimensions of the channels can be adjusted based on the desired droplet size. The droplets were collected at the outlet of the microfluidic device using a pipette tip. They were then placed inside a chamber that can house one million droplets (FIG. 5) and imaged using a fluorescent microscope and CCD camera.

The device was fabricated using soft lithography, as previously described (Mazutis et al. Nat. Protoc. 8(5):870-891, 2013). First, a transparent mask designed using a computer-aided design (CAD) was prepared. Next, a uniform layer of negative photoresist (SU-8) was spin coated on a silicon wafer, where the height was controlled by the spin rate. The mask was placed on top of the photoresist, and the pattern was transferred onto the photoresist using UV light to create a master mold. The wafer was then baked and developed. Next, polydimethylsiloxane (PDMS) pre-polymer was cast on the master mold and thermally cured. After several hours of incubation, the PDMS device was then cut and peeled off from the master mold and bound to a glass slide using a plasma machine. The surface of the device was then passivated using Aquapel glass treatment, which makes the device hydrophobic.

### B. Data Acquisition and Analysis

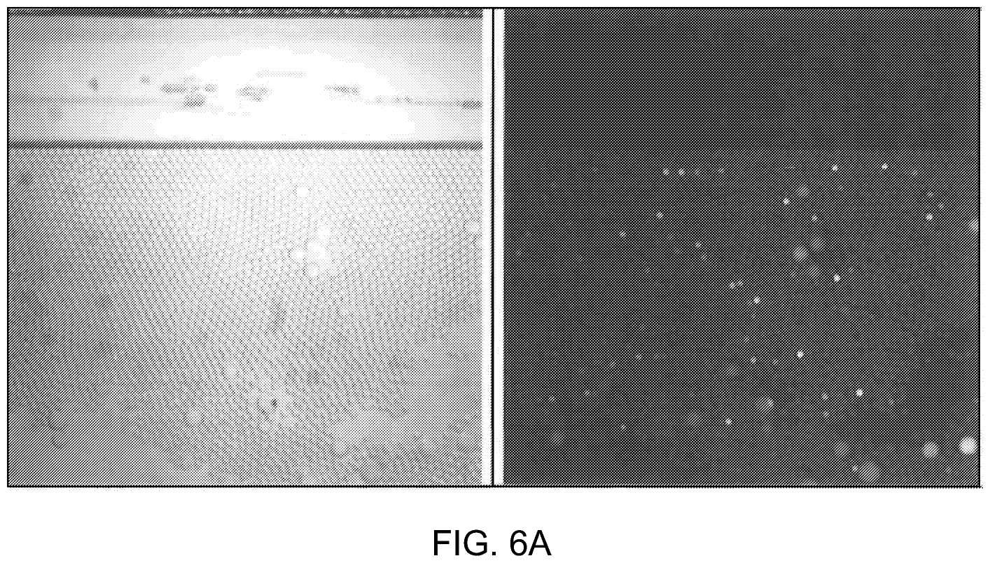

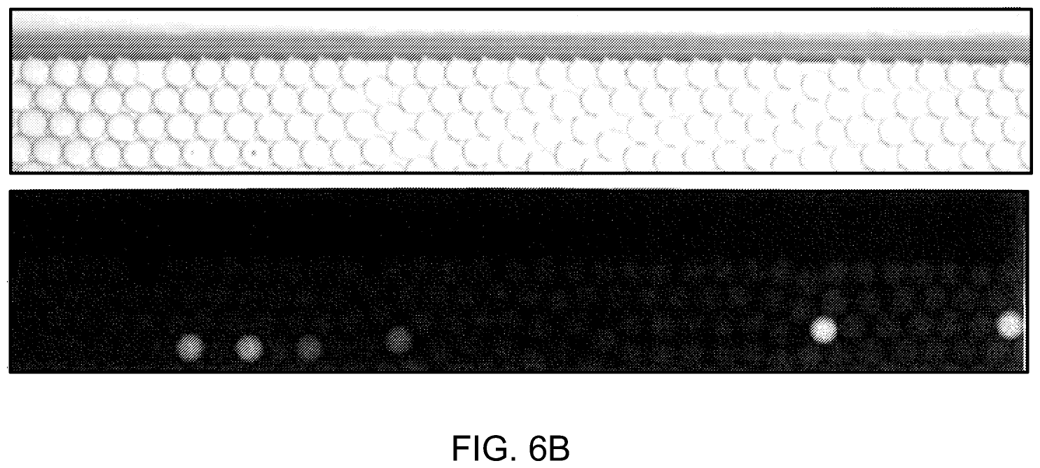

Droplets were imaged using a standard fluorescence microscope and a CCD camera. Representative images are shown in FIG. 6A. First, a white-light image was obtained to determine which droplets contain beads and which droplets are empty. Next, a fluorescent image was taken to determine which bead-containing droplets are labeled with an enzyme, and thus contain the fluorescent product. In the experiment shown in FIGS. 6A and 6B, excess enzyme was added such that each bead is bound to multiple enzymes. Thus, every droplet which contains a bead is expected to have a fluorescent signal (FIG. 6B).

### C. Multiplexed Analysis

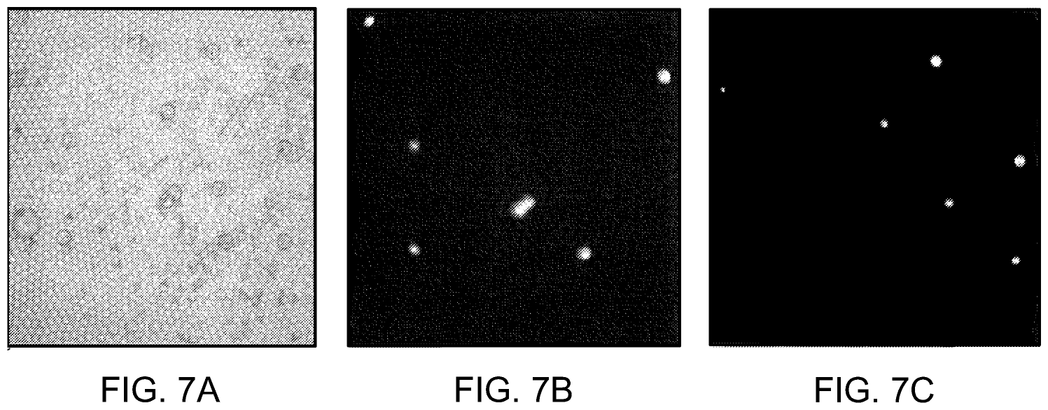

To assess whether optically distinct beads can be identified, two different dye-encoded beads (4′,6-diamidino-2-phenylindole (DAPI) and CY®7) were mixed, loaded into droplets, and then imaged. First, a white light image was obtained to locate the beads (FIG. 7A), and then two fluorescent images were obtained at the DAPI channel and the CY®7 channel (FIGS. 7B and 7C, respectively). As depicted in FIGS. 7B and 7C, the optically distinct beads were distinguishable. The results demonstrate that droplet arrays can be used for multiplexed detection of multiple analytes using optically encoded beads.

### D. Conclusion

Conventional ELISAs are not adequately sensitive to measure many proteins and other molecules in biological samples. To overcome limitations in analytical sensitivity, methods using smaller reaction volumes have been developed. However, these methods still have inadequate sensitivity and multiplexing capabilities. The current disclosure provides a novel approach for single molecule detection of proteins using a single molecule bead-based immunoassay and droplet microfluidics. This approach enables multiplexed and ultra-high sensitivity measurements of proteins and other biomolecules such as nucleic acids and metabolites.

### Example 5: A Mixing Mechanism Improves the Signal of Droplet Array Assays

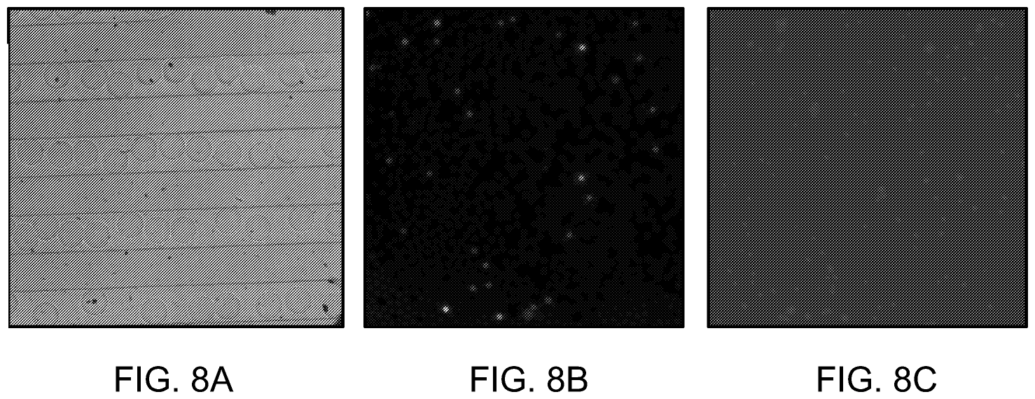

A mechanism to promote mixing was designed and its effect on signal of droplet array assays was evaluated. As depicted in FIGS. 8A-8C, channels with turns were used to promote mixing (e.g., mixing by chaotic advection) of the contents inside the droplets. In this example, droplets were formed and then passed through the mixing channels. The droplet generation configuration and channels for mixing were present on the same device. As depicted in FIGS. 8A-8C, the mixing mechanism improved the signal of the droplet array assay.

### Example 6: Device Configuration for Pre-mixing of Beads with Substrate

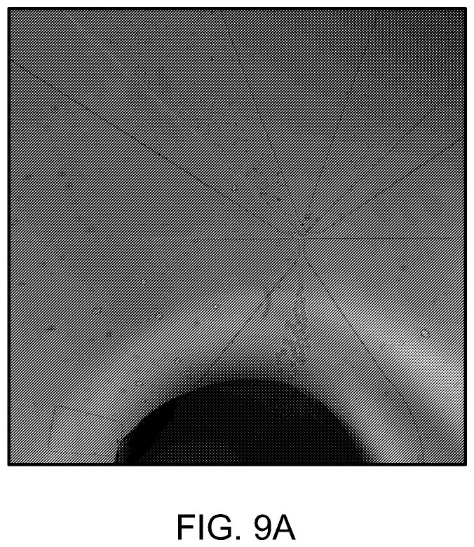

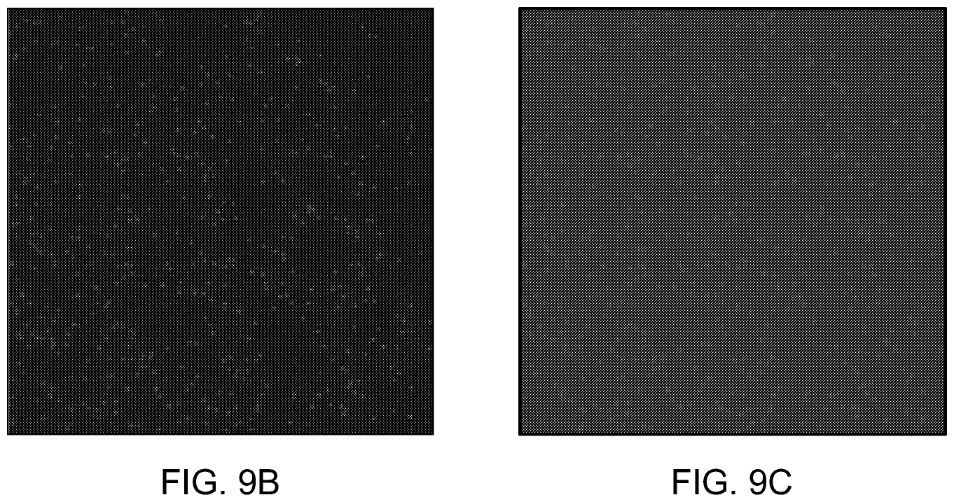

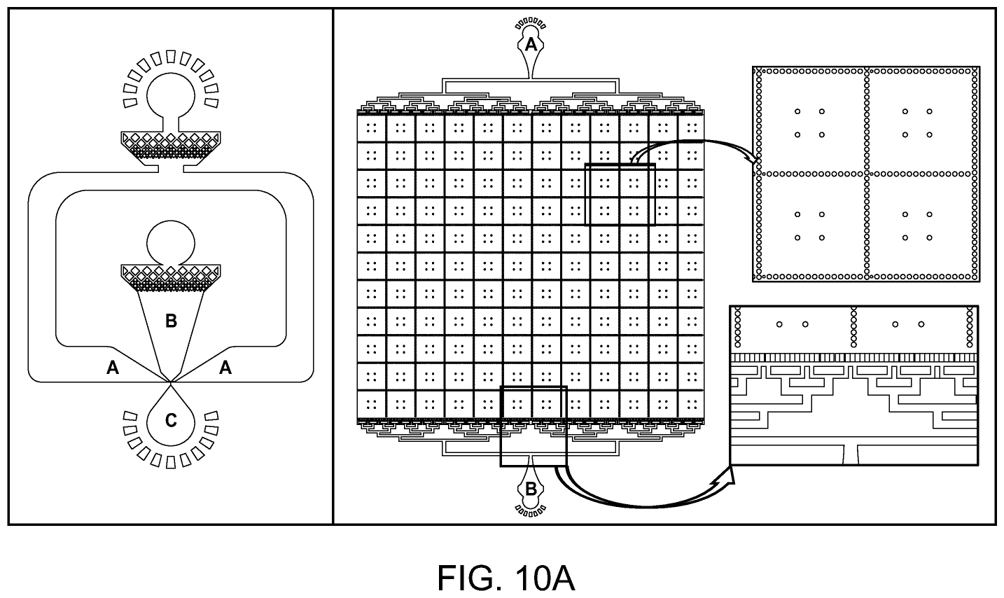

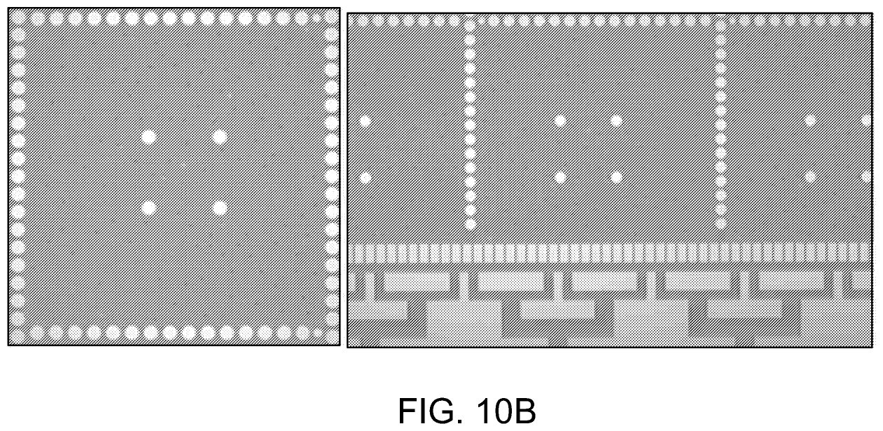

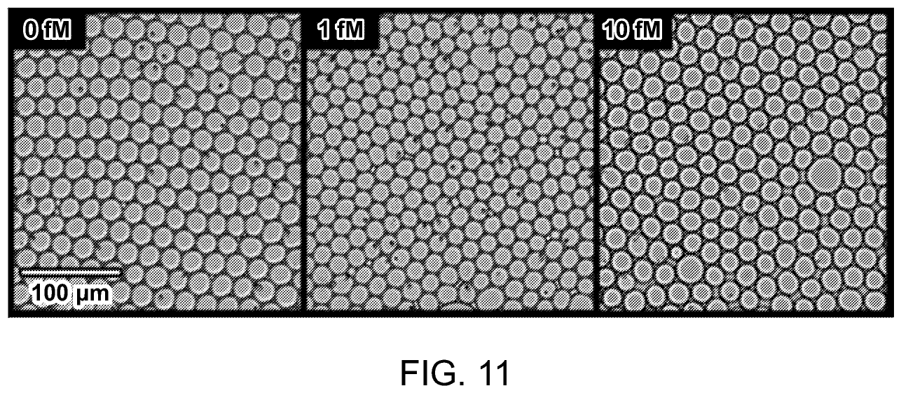

A microfluidic device configuration was designed in which the beads were pre-mixed with the substrate and then loaded onto a device for encapsulation inside droplets (FIGS. 9A-9C). The device design is simple and only requires inlets for beads and oil. In this configuration, a channel contained the beads in a solution of substrate. A second channel contained the oil. The beads were pre-mixed with the substrate and loaded into an inlet. In a second inlet, oil was added, following which the droplets were formed (FIGS. 10A and 10B). One advantage of this device configuration is a reduction in the number of droplets that are produced. For example, if the substrate is added in a separate channel and the total volume of the beads is 1 μl and the beads and substrate are added into a droplet in a 1:1 ratio by volume, the total volume is 2 μl. A higher volume means more droplets. The number of droplets that are generated can be reduced in half if the substrate is pre-mixed with the beads. Other advantages of this configuration include reduction in waste and reagent volume.

Specifically, to improve the sensitivity of the detection, a novel device was designed for efficiently generating droplets and packing them into an imaging chamber for analysis (FIGS. 10A and 10B). To be able to measure ultra-low levels of protein molecules, a major consideration is to ensure minimal sample loss at two different steps. The first is during droplet formation and the second is during loading of the droplets into the chamber. This will ensure adequate sampling of low numbers of molecules

Thus, when designing the droplet generating device, it is important to ensure droplet stability with minimal sample loss during droplet generation. In one example, a low input volume of 2 μl was used, which is a mixture of beads and substrate. An important consideration for the design of the droplet-generating device is to ensure droplet stability when the input volume is low, while still generating many droplets per second. Due to the low input volume (2 μl), it is difficult to ensure droplet stability from start of droplet generation. To generate many droplets per second, a pump-driven droplet generation system is preferred instead to vacuum driven system. However, pump-driven system often suffers from low droplet stability during the initial droplet generation. To solve this issue, ˜1 ul oil was added before the water phase in the sample tubing such that this oil will be injected first into the channel and stabilize the system prior to droplet generation.

Furthermore, to simplify the device and reduce imaging time, a minimal number of droplets were generated, while still ensuring that each droplet contains either zero beads or one bead for digital analysis. Since 100,000 beads were used for the assay, the number of droplets generated should be approximately 1 million. Therefore, the 2 μl volume is partitioned into pL-sized droplets. The droplet diameter is 14 μm (approximately 1.4 pL), which leads to about 1.4 million droplets. To ensure that the number of droplets was achieved, the input sample volume was fixed at about 2 μl. Therefore, the droplet generating device contains two inlets, one for the oil with surfactant and one for the beads and substrate mixture. In this device, the fluorogenic substrate and the beads were pre-mixed, and then added to the inlet to generate droplets. Using this approach, droplets are formed at approximately 10,000 droplets per second, for a total of two minutes. Due to the larger volume of the droplets (pL), compared to the volume of the traditional Simoa microwells (fL), background signal from pre-mixing is low. Following droplet generation, the droplets are then loaded onto a chamber for imaging.

The design of the imaging chamber, and the process of loading the droplets into the chamber were also optimized. To minimize droplet loss and maximize droplet packing within the imaging chamber, emulsions were loaded into a syringe tubing with the tip of the tubing points downward so that droplets can flow to the top of the emulsion after a few minutes due to gravity and low density of water phase comparing to the oil phase. Once packing is achieved, emulsions were injected into the imaging chamber. The imaging chamber was designed such that the droplets were packed in a monoloayer. As depicted in FIG. 10B, the distance between the two posts is 7 μm. Spacing between two posts is 15 μm, and the post diameter is 60 μm. Due to the shallow height (˜10 μm) of the microfluidics device, posts with 60 μm diameter were used throughout the device to prevent chamber collapse during microfluidics chip fabrication. To achieve good image quality, droplets need to be stationary for the entire duration of imaging. To achieve this, smaller posts with a spacing of 15 μm for each viewing area were designed so that droplets can squeeze through posts and simultaneously droplets were fixed in position after loading. A droplet blocking feature with 7 μm posts was designed near the outlet to prevent any droplet from escaping the device for maximum droplet capture efficiency. Using a combination of these features, maximum droplet loading and stationary droplet formation in a monolayer were achieved.

To analyze the arrays, droplets were first detected using the brightfield layer. Beads were then detected using the bead layer and assigned each bead to a droplet. The intensity in each droplet was then detected using the enzyme layer, and corrected for local background by subtracting the signal from the nearest droplets that do not contain a bead. This allowed for detection of the intensity of each bead containing droplets. In the digital range, the subtracted intensity for most bead containing droplets is zero. Furthermore, due to the digital nature of the assay, this setup is robust to variation in droplet size. Using this approach, up to 60% of the beads can be analyzed to achieve ultra-sensitive detection of target analytes.

### Example 7: Detection of Protein Targets Using Droplet Arrays

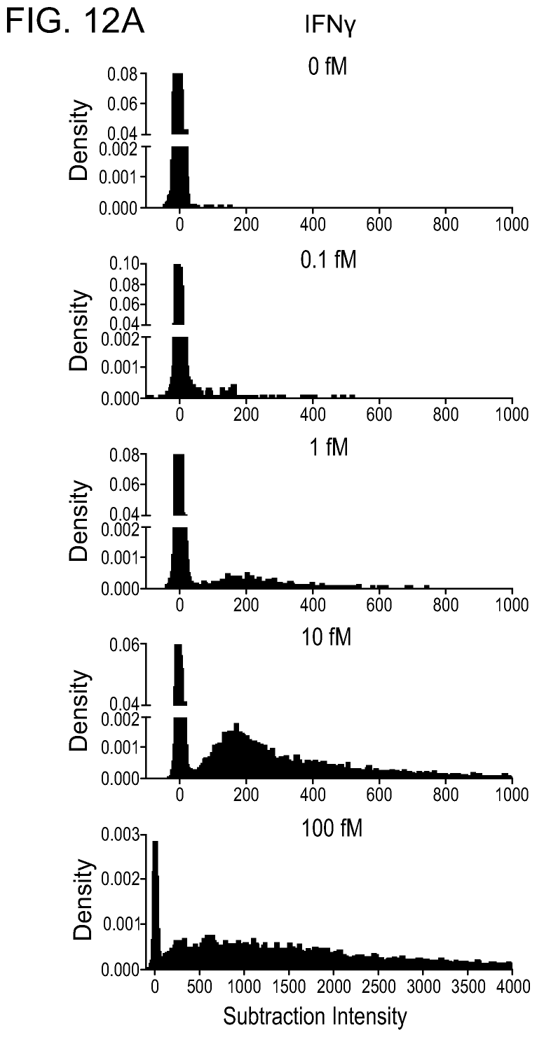

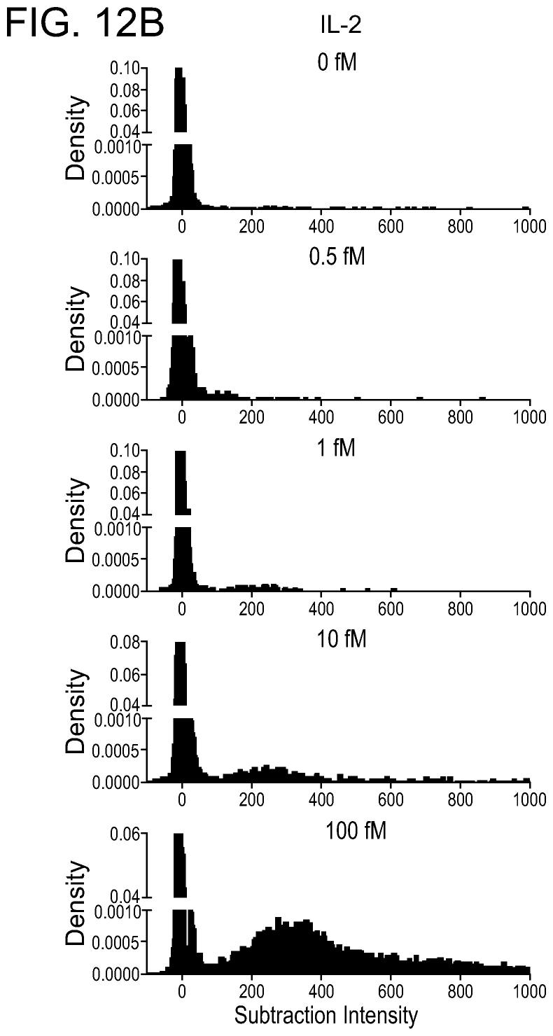

To evaluate the droplet array detection approach, two protein targets, IFNγ and IL-2, which are present at ultra-low levels in many biological samples were tested. Calibration curves were generated for both IFNγ and IL-2, with the lowest concentration of 0.0001 fM for IFNγ and 0.001 fM for IL-2. A blank sample for both proteins were also measured. The highest measured concentration was 100 fM for both assays. At low concentrations, most of the droplets contain no target protein molecule, and as the concentration increases, a small percentage of droplets contains more than one molecule. Representative histograms of the signal in the bead-containing droplets are shown for various concentrations in FIGS. 12A and 12B. As the concentration increases, a second population can be observed, indicating that some droplets may have more than one enzyme at higher concentrations.

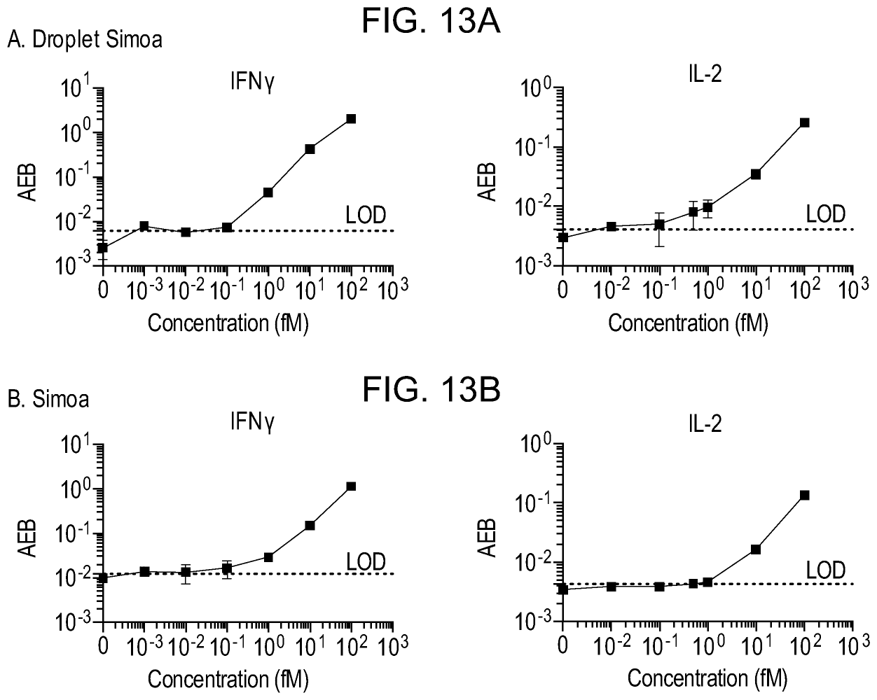

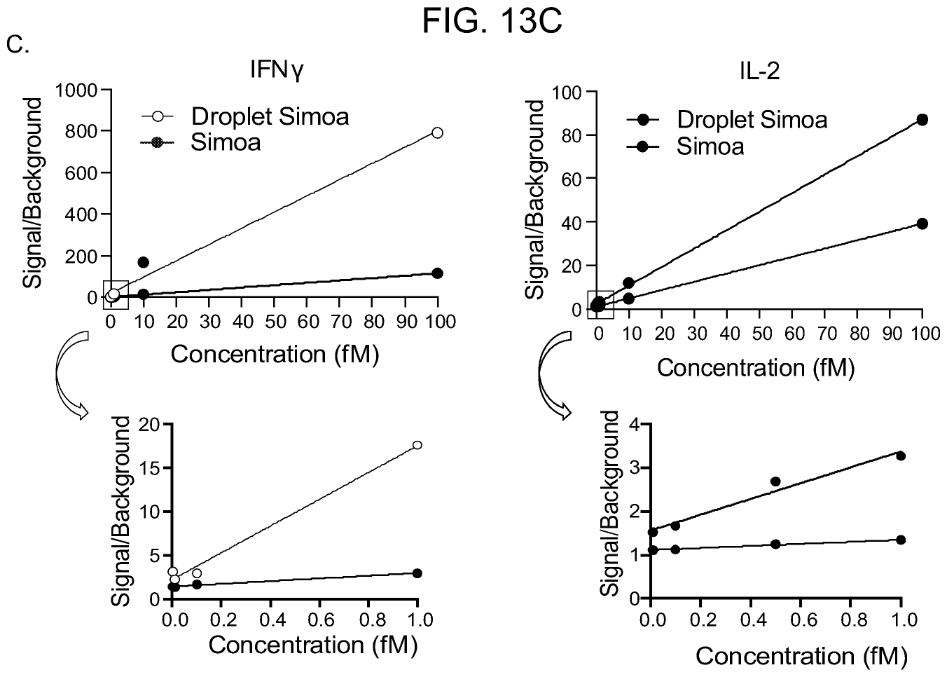

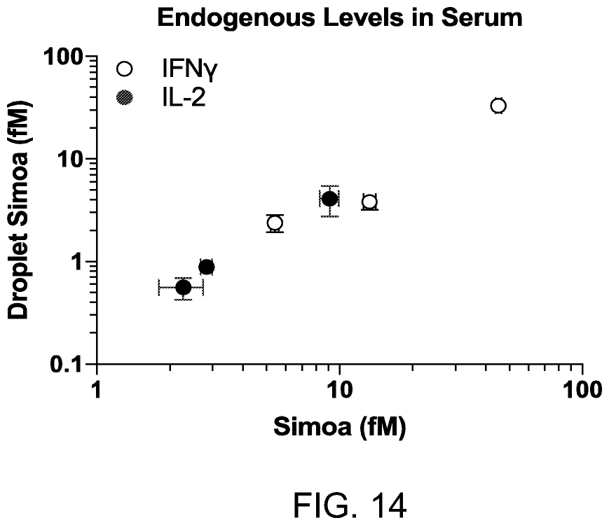

The full calibration curves are shown in FIGS. 13A-13C. The results were compared to the Simoa assay using the HD1 Analyzer (Quanterix) and the calibration curves are shown in FIG. 13B. Signal over the background were also calculated for both the droplet assay format and the Simoa assay format and a signal increase greater for the droplet assays compared to the Simoa assay was observed (FIG. 13C). The detection limits (LODs) and quantification limits (LOQs) for droplet assays were determined for Simoa assays, and the commercial Quanterix assay (Table 3) and show that sensitivity in the aM range can be achieved using the droplet-based approach, which is an approximately 25 fold improvement in the calculated detection limit over the Quanterix assay. Reducing the number of beads by five folds and increasing the number of beads analyzed allowed for the improvement in sensitivity. Finally, to ensure that the proteins can be detected reliably in serum, three serum samples were tested per marker and the results were compared to the calculated Quanterix values. The results from the two assays are in good agreement (FIG. 14). Thus, using the methods of the current invention, one can reliably measure proteins with ultra-high sensitivity over the current gold standard method for ultra-sensitive protein measurements.

TABLE 3
LODs and LOQs for Droplet Assays and Quanterix Assay

| | Droplet | Simoa | Quanterix | | Droplet | Simoa |
|---|---|---|---|---|---|---|
| | Simoa LOD | LOD | LOD | | Simoa LOQ | LOQ |
| | (3X) | (3X) | (2.5X) | | (10X) | (10X) |
| IFNγ | 30 aM | 350 aM | 1.00 | fM | 260 aM | 1.24 fM |
| IL-2 | 20 aM | 550 aM | 730 | aM | 360 aM | 2.17 fM |

The approach in this example is also amenable to other single molecule studies that are not based on bead-based immunoassays. One example is detection of rare enzyme molecules in blood. Currently, the single molecule microwell array has about 216,000 wells, and each well can hold 50 fLs in volume. Therefore, the total volume that can be interrogated is 10.8 nL. Since the volume that must be loaded onto the array is 15 μl, the vast majority of the sample cannot be interrogated, and thus, detection of rare molecules that are not bound to a bead is not possible. Using the novel approach in this example, it is possible to interrogate sample volumes at the μl level, which enables single molecule detection of low abundance target molecules.

What is claimed is:

1. A method of detecting a target analyte in a sample, the method comprising the steps of:
(a) contacting a sample containing or suspected of containing a target analyte with a plurality of capture probes, the capture probes being linked to one or more capture ligands that specifically bind to the target analyte, and incubating to allow binding of the capture ligands to the target analytes;
(b) contacting the product of (a) with a plurality of detection probes that specifically bind to the target analyte, and incubating to allow binding of the detection probes to the target analyte, the detection probes each being linked to a detectable moiety;
(c) producing a plurality of droplets from the product of (b); and
(d) detecting the detectable moieties present in the plurality of droplets, thereby detecting the target analyte in the sample.

2. A method of detecting a target analyte in a sample, the method comprising the steps of:
(a) contacting a sample containing or suspected of containing a target analyte with: (i) a plurality of detection probes that specifically bind to the target analyte, and (ii) a plurality of capture probes, the capture probes being linked to one or more immobilized target analytes, wherein the detection probes competitively bind to the target analytes contained in the sample and to the immobilized target analytes;
(b) incubating the product of step (a) to allow binding of the detection probes to the target analytes contained in the sample or to the immobilized target analytes;
(c) labeling the detection probes that are bound to the immobilized target analytes linked to the capture probes of step (b) with detectable moieties;
(d) producing a plurality of droplets of the product of step (c); and
(e) detecting the detectable moieties in the droplets, thereby detecting the target analyte in the sample.

3. The method of claim 1 or 2, wherein all or substantially all of the droplets contain zero or one capture probes.

4. The method of claim 1 or 2, wherein all or substantially all of the droplets contain more than one capture probe.

5. The method of claim 1, 3, or 4, wherein steps (a) and (b) are performed sequentially or simultaneously.

6. The method of any one of claims 2-4, wherein steps (a), (b), and (c) are performed sequentially or simultaneously.

7. The method of any one of claims 1 or 3-5, wherein the capture probes are linked to about 1 to about 10¹² capture ligands.

8. The method of any one of claims 1-7, wherein the concentration of the target analyte in the sample ranges from about 0 aM to about 1 mM.

9. The method of any one of claims 1-8, wherein the droplets have a volume of about 0.01 pL to about 10 nL.

10. The method of any one of claims 1-9, wherein producing a plurality of droplets is performed using a microfluidic device.

11. The method of claim 10, wherein the microfluidic device is a flow-focusing device.

12. The method of any one of claims 1, 3-5, and 7-11, wherein the capture ligand and/or the detection probe is an antibody, an aptamer, an antibody mimetic, a polypeptide, a nucleic acid, a molecularly-imprinted polymer, a receptor, or a small molecule.

13. The method of any one of claims 1, 3-5, and 7-12, wherein the capture ligand and the detection probe are the same antibody, aptamer, antibody mimetic, polypeptide, nucleic acid, molecularly-imprinted polymer, receptor, or small molecule.

14. The method of any one of claims 1, 3-5, and 7-12, wherein the capture ligand and the detection probe are different antibodies, aptamers, antibody mimetics, polypeptides, nucleic acids, molecularly-imprinted polymers, receptors, or small molecules.

15. The method of claim 14, wherein the antibody is a full-length antibody or an antigen-binding antibody fragment.

16. The method of claim 15, wherein the full-length antibody is an IgG, IgA, IgD, IgE, or IgM antibody.

17. The method of claim 15, wherein the antigen-binding antibody fragment is an scFv, an Fv, a dAb, a Fab, an Fab′, an Fab′₂, an F(ab)₂, an Fd, an Fv, or an Feb.

18. The method of claim 14, wherein the antibody mimetic is an affibody, an affilin, an affimer, an affitin, an alphabody, an anticalin, an avimer, a DARPin, a fynomer, a Kunitz domain peptide, a monobody, or a nanoCLAMP.

19. The method of any one of claims 1-18, wherein the capture probes are selected from the group consisting of beads, nanotubes, or polymers.

20. The method of claim 19, wherein the beads are paramagnetic beads, silica beads, or hydrogel beads.

21. The method of claim 19 or 20, wherein the beads have a size of about 1 μm to about 50 μm.

22. The method of any one of claims 1-21, wherein in step (a), the sample is contacted with about 10,000 to about 100,000,000 capture probes.

23. The method of any one of claims 1-22, wherein the detectable moiety is or comprises an enzymatic label, a fluorescent label, a radioactive label, or a metal label.

24. The method of claim 23, wherein the detectable moiety is or comprises an enzymatic label.

25. The method of claim 24, wherein the enzymatic label is selected from the group consisting of beta-galactosidase, horseradish peroxidase, glucose oxidase, and alkaline phosphatase.

26. The method of claim 25, wherein the plurality of droplets are formed with an enzyme substrate.

27. The method of claim 26, wherein the enzyme substrate is di-β-D-galactopyranoside (FDG).

28. The method of claim 27, wherein the enzyme substrate is pre-mixed with the capture probes in step (a).

29. The method of any one of claims 1-28, wherein producing the droplets comprises mixing by chaotic advection

30. The method of claim 29, wherein the mixing by chaotic advection is performed using channels with turns in a microfluidic device.

31. The method of any one of claims 1-30, wherein the droplets comprise a density gradient medium.

32. The method of any one of claims 1-31, wherein the sample comprises a biological sample or an environmental sample.

33. The method of any one of claims 1-32, wherein the target analyte is a protein, a nucleic acid, a polysaccharide, a lipid, an extracellular vesicle, a glycan, a toxin, a cell, a fatty acid, a therapeutic agent, a pathogen, an organism, a virus, or a small molecule.

34. The method of claim 33, wherein the nucleic acid is a modified nucleic acid or an miRNA.

35. The method of claim 32, wherein the biological sample is a body fluid.

36. The method of claim 35, wherein the body fluid is selected from the group consisting of lymph, whole blood, plasma, serum, a blood fraction containing peripheral blood mononuclear cells, urine, saliva, semen, sweat, lacrimal fluid, synovial fluid, cerebrospinal fluid, feces, mucous, vaginal fluid, and spinal fluid.

37. The method of claim 32, wherein the biological sample is a breast tissue, a renal tissue, a colonic tissue, a brain tissue, a muscle tissue, a synovial tissue, skin, a hair follicle, bone marrow, a tumor tissue, a tissue lysate or homogenate, or an organ lysate or homogenate.

38. The method of any one of claims 1-37, wherein the detection comprises single-molecule detection of the detectable moieties.

39. The method of any one of claims 1-38, further comprising detecting an additional target analyte in the sample.

40. The method of claim 39, wherein the additional target analyte is a protein, a nucleic acid, a polysaccharide, a lipid, a cell, a fatty acid, a therapeutic agent, an organism, a virus, or a small molecule.

41. A method of detecting a first target analyte and a second target analyte in a sample, the method comprising the steps of:
(a) contacting a sample containing or suspected of containing a first target analyte and a second target analyte with:
(i) a plurality of first capture probes, the first capture probes being linked to one or more first capture ligands that specifically bind to the first target analyte; and
(ii) a plurality of second capture probes, the second capture probes being linked to one or more second capture ligands that specifically bind to the second target analyte,
and incubating to allow binding of the first and second capture ligands to the first and second target analytes, respectively;
(b) contacting the product of (a) with:
(i) a plurality of first detection probes that specifically bind to the first target analyte, and
(ii) a plurality of second detection probes that specifically bind to the second target analyte,
and incubating to allow binding of the first and second detection probes to the first and second target analytes, respectively, wherein the first and second detection probes are each labelled with a detectable moiety;
(c) producing a plurality of droplets from the product of (b); and
(d) detecting the detectable moieties present in the plurality of droplets, thereby detecting the first target analyte and the second target analyte in the sample.

42. The method of claim 41, wherein the first capture probe and the second capture probe are detectably and distinguishably labeled, and step (d) comprises detecting the capture probes and the detectable moieties present in the plurality of droplets.

43. The method of claim 41, wherein the first capture probe is labelled with a first dye, and the second capture probe is labelled with a second dye.

44. The method of any one of claims 41-43, wherein the first detection probe is labelled with a first detectable moiety, and the second detection probe is labelled with a second detectable moiety, and the first detectable moiety and the second detectable moiety are distinguishable.

45. The method of any one of claims 41-43, wherein the first detection probe is labelled with a first detectable moiety, and the second detection probe is labelled with a second detectable moiety, and the first detectable moiety and the second detectable moiety are not distinguishable.

46. The method of any one of claims 1-45, wherein all or substantially all of the droplets contain zero or one target analyte molecule.

47. The method of any one of claims 1-46, wherein the detectable moieties are detected in at least 30% of the droplets.

48. The method of any one of claims 1-46, wherein the detectable moieties are detected in at least 60% of the droplets.

47. The method of any one of claims 1-46, wherein the detectable moieties are detected in at least 90% of the droplets.
